(19) United States
(12) Patent Application Publication
(10) Pub. No.: US 2017/0052204 A1
(43) Pub. Date: Feb. 23, 2017

(54) Metabolic and Genetic Biomarkers for Memory Loss

(71) Applicants: GEORGETOWN UNIVERSITY, Washington, DC (US); UNIVERSITY OF ROCHESTER, Rochester, NY (US)

(72) Inventors: Howard J. Federoff, Bethesda, MD (US); Mark E. Mapstone, Pittsford, NY (US); Amrita K. Cheema, Potomac, MD (US); Massimo S. Fiandaca, Irvine, CA (US)

(21) Appl. No.: 15/307,988

(22) PCT Filed: Apr. 30, 2015
(86) PCT No.: PCT/US15/28550

§ 371 Date: Oct. 31, 2016

Related U.S. Application Data
(60) Provisional application No. 61/986,555, filed on Apr. 30, 2014.

(51) Int. Cl. G01N 33/92; C12Q 1/68

(52) U.S. Cl. CPC G01N 33/92; C12Q 1/6883; C12Q 2600/158; G01N 2570/00; G01N 2800/2814; G01N 2800/52

(57) ABSTRACT

The present invention relates to methods of determining if a subject has an increased risk of suffering from memory impairment. The methods comprise analyzing at least one plasma sample from the subject to determine a value of the subject's lipidomic profile, and also analyzing the gene expression profile from leukocytes and comparing the value of the subject's biomarker profile (lipidomic profile plus gene expression profile) with the value of a normal biomarker profile. A change in the value of the subject's biomarker profile, including a change in the subject's biomarker profile, over normal values is indicative that the subject has an increased risk of suffering from memory impairment compared to a normal individual.

### STATEMENT REGARDING FEDERALLY SPONSORED RESEARCH OR DEVELOPMENT

Part of the work performed during development of this invention utilized U.S. Government funds under National Instituted of Health Grant No. R01 AG030753 and Department of Defense Contract No. W81XWH-09-1-0107. The U.S. Government has certain rights in this invention.

### BACKGROUND OF THE INVENTION

Field of the Invention

The present invention relates to methods of determining if a subject has an increased risk of suffering from memory impairment. The methods comprise analyzing at least one plasma sample from the subject to determine a value of the subject's lipidomic profile, and also analyzing the gene expression profile from leukocytes and comparing the value of the subject's biomarker profile (lipidomic profile plus gene expression profile) with the value of a normal biomarker profile. A change in the value of the subject's biomarker profile, including a decrease in the subject's lipidomic profile, over normal values is indicative that the subject has an increased risk of suffering from memory impairment compared to a normal individual.

Background of the Invention

Alzheimer's disease (AD) is a neurodegenerative disorder characterized by a progressive dementia that insidiously and inexorably robs older adults of their memory and other cognitive abilities. The prevalence of AD is expected to double every 20 years from 35.6 million individuals worldwide in 2010 to 115 million affected individuals by 2050. There is no cure and current therapies are unable to slow the disease progression.

Early detection of the at-risk population (preclinical), or those in the initial symptomatic stages (prodromal) of AD, may present opportunities for more successful therapeutic intervention, or even disease prevention by interdicting the neuropathological cascade that is ultimately characterized by the deposition of extracellular β-amyloid (Aβ) and accumulation of intracellular fibrils of microtubular hyperphosphorylated tau protein within the brain. Biomarkers for early disease, including cerebrospinal fluid (CSF) tau and Aβ levels, structural and functional magnetic resonance imaging (MRI), and the recent use of brain positron emission tomography (PET) amyloid imaging, are of limited use as widespread screening tools since they provide diagnostic options that are either invasive (i.e., require lumbar puncture), time-consuming (i.e., several hours in a scanner for most comprehensive imaging protocols), or expensive. No current blood-based biomarkers can detect incipient dementia with the required sensitivity and specificity during the preclinical stages. Continued interest in blood-based biomarkers remains because these specimens are obtained using minimally invasive, rapid, and relatively inexpensive methods. With recent technological advances in ‘omics’ technologies and systems biology analytic approaches, the comprehensive bioinformatic analyses of blood-based biomarkers may not only yield improved accuracy in predicting those at risk, but may also provide new insights into the underlying mechanisms and pathobiological networks involved in AD and possibly herald the development of new therapeutic strategies.

The preclinical interval resulting in mild cognitive impairment (MCI) or AD is known to be variable, multifactorial, and extends for at least 7-10 years prior to the emergence of clinical signs. In the absence of accurate and easily obtained biomarkers, multimodal neurocognitive testing remains the most accurate, standardized, and widely used pre-mortem screening method to determine the presence or absence of clinical MCI or AD. The utility of strict cognitive assessment for preclinical stages of MCI or AD is limited, however, as this approach is not only time-consuming but is expected, by definition, to be normal in preclinical subjects. Neuropsychological testing is able to quantitatively delineate specific brain alterations from normal, such as memory, attention, language, visuoperceptual, and executive functions, which are typically not affected in individuals during the preclinical stages. Thus, information obtained from multiple diagnostic studies will probably be most useful in defining the MCI/AD preclinical stages, including neuropsychological testing and some form(s) of biomarker(s). While CSF and neuroimaging have been used to define preclinical MCI/AD to date, their clinical utility as screening tools for asymptomatic individuals is not established.

### SUMMARY OF THE INVENTION

The present invention relates to methods of determining if a subject has an increased risk of suffering from memory impairment. In one embodiment the subject is cognitively unimpaired prior to determining the risk of impairment. The methods comprise analyzing at least one plasma sample from the subject to determine a value of the subject's lipidomic profile, and also analyzing the gene expression profile from leukocytes and comparing the value of the subject's biomarker profile (lipidomic profile plus gene expression profile) with the value of a normal biomarker profile. A change in the value of the subject's biomarker profile, including a decrease in the subject's lipidomic profile, compared to normal values is indicative that the subject has an increased risk of progressing to or suffering from memory impairment compared to a normal individual.

### BRIEF DESCRIPTION OF THE DRAWINGS

FIG. 1 depicts box and whisker plots of the combined discovery and validation samples on the five composite cognitive Z-score measures. The performance of the Converter group before (Cₚᵣₑ) after (Cₚₒₛₜ) phenoconversion is plotted for direct comparison to Cognitively normal subjects (NC) and those with clinically evident disease (MCI/AD). The blue line centered on 0 in each plot represents the median Z-score on that measure for the entire cohort. The horizontal black line in each plot represents the cut-off for impairment (−1.35 SD). Error bars represent s.e.m. Note that while minimal declines were seen in all neurocognitive spheres, the most significant change from Cₚᵣₑ to Cₚₒₛₜ occurred with the Zₘₑₘ, where all converters had non-impaired memory a baseline and significantly impaired memory after phenoconversion. NC=Normal Control (n=73); Cₚᵣₑ=Converters at baseline, prior to phenoconversion, (n=28); Cₚₑₛₜ=Converters after phenoconversion (n=28); MCI/AD=mild cognitive impairment or AD (n=46).

FIG. 2 depicts the quantitative profiling of the data. Specifically, the SID-MRM-MS (stable isotope dilution-multiple reaction monitoring mass spectrometry) based quantitative profiling data were subjected to the non-parametric Kruskal Wallis test using the STAT pack module (Biocrates Inc.). Results are shown for a panel of ten metabolites in the NC group (n=53), Cₚᵣₑ (n=18), Cₚₒₛₜ (n=18) and aMCI/AD (n=35) groups, respectively. The abundance of each metabolite was plotted as normalized concentrations units (nM). The black solid bars within the boxplot represent the median abundance, and the dotted line represents mean abundance for the given group. Error bars represent ±s.d. QC, quality control samples. The P values for analytes between groups were P 0.05. The two metabolites with P values <0.005 are indicated with an asterisk. Each Kruskal-Wallis test was followed by Mann-Whitney U-tests for post hoc pairwise comparisons (NC versus Cₚᵣₑ and NC versus aMCI/AD). Significance was adjusted for multiple comparisons using Bonferroni's method (P<0.025).

FIG. 3 depicts box plots for the ten metabolite panel validation study. This figure shows the results of the blinded, internal cross-validation for each of the ten metabolites using targeted, quantitative mass spectrometry. The solid line represents the median abundance for the given group and the dotted line represents mean abundance. The three randomly assigned blinded groups (A, B, C), were predicted to include NC (n=20) as group C, Cₚᵣₑ (n=10) as group A, and aMCI/AD (n=20) as group B. These predictions, based on the quantitative measures, were confirmed when the blind was lifted following the analysis. QC depicts the range in the quality control samples.

FIG. 4 depicts receiver operating characteristic (ROC) curve results for the lipidomics analyses. (a-c) Plots of ROC results from the models derived from the three phases of the lipidomics analysis. Simple logistic models using only the metabolites identified in each phase of the lipidomics analysis were developed and applied to determine the success of the models for classifying the Cᵣ, and NC groups. The red line in each plot represents the AUC obtained from the discovery-phase LASSO analysis (a) the targeted analysis of the ten metabolites in the discovery phase (b) and the application of the ten-metabolite panel developed from the targeted discovery phase in the independent validation phase (c). The ROC plots represent sensitivity (i.e., true positive rate) versus 1−specificity (i.e., false positive rate).

FIG. 5 depicts the receiver operating characteristic (ROC) area under the curve (AUC) of the multimodal classifier model used to differentiate cognitively normal individuals who will phenoconvert to aMCI/AD within 2-3 years (Cₚᵣₑ) from a group of cognitively normal (NC) individuals who will remain cognitively normal for the next 2-3 years. The multimodal classifier model used in this case utilizes the combination of 10 lipids (Table 1) and 9 genes (Table 2). The classifier model has a 99.8% accuracy for the correct classification between the Cₚᵣₑ and NC groups based solely on these lipids and genes.

### DETAILED DESCRIPTION OF THE INVENTION

The present invention relates to methods of determining if a subject has an increased risk of suffering from memory impairment. The methods comprise analyzing at least one plasma sample from the subject to determine a value of the subject's lipidomic profile, and also analyzing the gene expression profile from leukocytes and comparing the value of the subject's biomarker profile (lipidomic profile plus gene expression profile) with the value of a normal biomarker profile. A change in the value of the subject's biomarker profile, including a decrease in the subject's lipidomic profile, over normal values is indicative that the subject has an increased risk of suffering from memory impairment compared to a normal individual.

As used herein, the term subject or “test subject” indicates a mammal, in particular a human or non-human primate. The test subject may or may not be in need of an assessment of a predisposition to memory impairment. For example, the test subject may have a condition or may have been exposed to injuries or conditions that are associated with memory impairment prior to applying the methods of the present invention. In another embodiment, the test subject has not been identified as a subject that may have a condition or may have been exposed to injuries or conditions that are associated with memory impairment prior to applying the methods of the present invention.

As used herein, the phrase “memory impairment” means a measureable or perceivable decline or decrease in the subject's ability to recall past events. As used herein, the term “past events” includes both recent (new) events (short-term memory) or events further back in time (long-term memory). In one embodiment, the methods are used to assess an increased risk of short-term memory impairment. In another embodiment, the methods are used to assess an increased risk in long-term memory impairment. The memory impairment can be age-related memory impairment. The memory impairment may also be disease-related memory impairment. Examples of disease-related memory impairment include but are not limited to Alzheimer's Disease, Parkinson's Disease, Multiple Sclerosis, Huntington's Disease, Pick's Disease, Progressive Supranuclear Palsy, Brain Tumor(s), Head Trauma, and Lyme Disease to name a few. In one embodiment, the memory impairment is related to amnestic mild cognitive impairment (aMCI). In another embodiment, the memory impairment is related to Alzheimer's Disease. The root cause of the memory impairment is not necessarily critical to the methods of the present invention. The measureable or perceivable decline in the subject's ability to recall past events may be assessed clinically by a health care provider, such as a physician, physician's assistant, nurse, nurse practitioner, psychologist, psychiatrist, hospice provider, or any other provider that can assess a subject's memory. The measureable or perceivable decline in the subject's ability to recall past events may be assessed in a less formal, non-clinical manner, including but not limited to the subject himself or herself, acquaintances of the subject, employers of the subject and the like. The invention is not limited to a specific manner in which the subject's ability to recall past events is assessed. In fact, the methods of the invention can be implemented without the need to assess a subject's ability to recall past events. Of course, the methods of the present invention may also include assessing the subject's ability to assess past events one or more times, both before determining the subject's biomarker profile (lipidomic profile and gene expression profile) after determining the subject's biomarker profile (lipidomic profile and gene expression profile) at least one time.

In one embodiment, the decline or decrease in the ability to recall past events is relative to each individual's ability to recall past events prior to the diagnosed decrease or decline in the ability to recall past events. In another embodiment, the decline or decrease in the ability to recall past events is relative to a population's (general, specific or stratified) ability to recall past events prior to the diagnosed decrease or decline in the ability to recall past events.

As used herein, the term means “increased risk” is used to mean that the test subject has an increased chance of developing or acquiring memory impairment compared to a normal individual. The increased risk may be relative or absolute and may be expressed qualitatively or quantitatively. For example, an increased risk may be expressed as simply determining the subject's biomarker profile (lipidomic profile and gene expression profile) and placing the patient in an “increased risk” category, based upon previous population studies. Alternatively, a numerical expression of the subject's increased risk may be determined based upon the biomarker profile (lipidomic profile and gene expression profile). As used herein, examples of expressions of an increased risk include but are not limited to, odds, probability, odds ratio, p-values, attributable risk, relative frequency, positive predictive value, negative predictive value, and relative risk.

For example, the correlation between a subject's biomarker profile (lipidomic profile and gene expression profile) and the likelihood of suffering from memory impairment may be measured by an odds ratio (OR) and by the relative risk (RR). If P(R) is the probability of developing memory impairment for individuals with the risk profile (R) and P(R) is the probability of developing memory impairment for individuals without the risk profile, then the relative risk is the ratio of the two probabilities: RR=P(R⁺)/P(R⁻).

In case-control studies, however, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of a lipidomic risk profile in cases studies and F is the frequency of lipidomic risk profile in controls. F⁺ and F⁻ can be calculated using the biomarker profile (lipidomic profile and gene expression profile) frequencies of the study.

The attributable risk (AR) can also be used to express an increased risk. The AR describes the proportion of individuals in a population exhibiting memory impairment due to a specific member of a lipidomic risk profile or a specific member of the gene expression profile. AR may also be important in quantifying the role of individual components (specific member) in disease etiology and in terms of the public health impact of the individual marker. The public health relevance of the AR measurement lies in estimating the proportion of cases of memory impairment in the population that could be prevented if the profile or individual component were absent. AR may be determined as follows: AR=P_{E}(RR−1)/(P_{E}(RR−1)+1), where AR is the risk attributable to a profile or individual component of the profile, and P_{E} is the frequency of exposure to a profile or individual component of the profile within the population at large. RR is the relative risk, which can be approximated with the odds ratio when the profile or individual component of the profile under study has a relatively low incidence in the general population.

In one embodiment, the increased risk of a patient can be determined from p-values that are derived from association studies. Specifically, associations with specific profiles can be performed using regression analysis by regressing the biomarker profile (lipidomic profile and gene expression profile) with memory impairment. In addition, the regression may or may not be corrected or adjusted for one or more factors. The factors for which the analyses may be adjusted include, but are not limited to age, sex, weight, ethnicity, geographic location, fasting state, state of pregnancy or post-pregnancy, menstrual cycle, general health of the subject, alcohol or drug consumption, caffeine or nicotine intake and circadian rhythms, and the subject's apolipoprotein epsilon (ApoE) genotype to name a few.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses are conducted with the program SAS.

SAS (“statistical analysis software”) is a general purpose package (similar to Stata and SPSS) created by Jim Goodnight and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis.

As used herein, the phrase “biomarker profile” means the combination of a subject's lipidomic profile and the subject's gene expression profile.

As used herein, the phrase “lipidomic profile” means a collection of measurements, such as but not limited to a quantity or concentration, for individual lipid molecules taken from a test sample of the subject. Examples of test samples or sources of components for the lipidomic profile include, but are not limited to, biological fluids, which can be tested by the methods of the present invention described herein, and include but are not limited to whole blood, such as but not limited to peripheral blood, serum, plasma, cerebrospinal fluid, urine, amniotic fluid, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue.

As used herein, the phrase “gene expression profile” means a collection of measurements, such as but not limited to a quantity or concentration, for expression of individual genes taken from the RNA or protein extracts of a test sample of the subject. Examples of test samples or sources of components for the RNA or protein extracts for the gene expression profile include, but are not limited to, biological fluids, such as but not limited to whole blood, serum, plasma, cerebrospinal fluid, urine, amniotic fluid, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue. In specific embodiments, RNA or protein extracts from cells that are contained in the samples are used to generate a gene expression profile.

Techniques to assay levels of individual components of the lipidomic profile from test samples are well known to the skilled technician, and the invention is not limited by the means by which the components are assessed. In one embodiment, levels of the individual components of the lipidomic profile are assessed using mass spectrometry in conjunction with ultra-performance liquid chromatography (UPLC), high-performance liquid chromatography (HPLC), and UPLC to name a few. Other methods of assessing levels of the individual components include biological methods, such as but not limited to ELISA assays.

The assessment of the levels of the individual components of the lipidomic profile can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the individual components of the lipidomic profile, a sample is taken from the subject. The sample may or may not processed prior assaying levels of the components of the lipidomic profile. For example, whole blood may be taken from an individual and the blood sample may be processed, e.g., centrifuged, to isolate plasma or serum from the blood. The sample may or may not be stored, e.g., frozen, prior to processing or analysis.

Individual components of the lipidomic profile include but are not limited to phosphatidyl cholines (PC) lyso PCs and acylcarnitines (AC). Specific examples of PCs, lyso PCs and ACs that can be included as constituents of the lipidomic profile include but are not limited to (1) propionyl AC, (2) lyso PC a C18:2, (3) PC aa C36:6, (4) C16:1-OH (Hydroxyhexadecenoyl-L-carnitine), (5) PC aa C38:0, (6) PC aa 36:6, (7) PC aa C40:1, (8) PC aa C40:2, (9) PC aa C40:6 and (10) PC ae C40:6. Those of skill in the art will recognize the specific identity of each constituent listed based upon the nomenclature above. For example, metabolite (5) (PC aa C38:0) is known to those of skill in the art as phosphatidylcholine diacyl C 38:0, metabolite (10) (PC ae C40:6) is known as phosphatidylcholine acyl-alkyl C 40:6 and metabolite (2) (lyso PC a C18:2) is known as lysoPhosphatidylcholine acyl C18:2. In one embodiment, the individual levels of each of the lipid metabolites are lower than those compared to normal levels. In another embodiment, one, two, three, four, five, six, seven, eight or nine of the levels of each of the lipid metabolites are lower over normal levels.

The levels of depletion of the lipids over normal levels can vary. In one embodiment, the levels of (1) propionyl AC are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (2) lyso PC a C18:2 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (3) PC aa C36:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (4) C16:1-OH (Hydroxyhexadecenoyl-L-carnitine), are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (5) PC aa C38:0 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (6) PC aa 36:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (7) PC aa C40:1 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (8) PC aa C40:2 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (9) PC aa C40:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (10) PC ae C40:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. For the purposes of the present invention, the number of “times” the levels of a metabolite is lower or higher over normal can be a relative or absolute number of times. In the alternative, the levels of the metabolites may be normalized to a standard and these normalized levels can then be compared to one another to determine if a metabolite is lower or higher.

For the purposes of the present invention the lipidomic profile comprises at least two, three, four, five, six, seven, eight, nine or all ten metabolites listed above. If two metabolites are used in generating the lipidomic profile, any combination of two of 1-10 listed above can be used. If three metabolites are used in generating the lipidomic profile, any combination of three of 1-10 listed above can be used. If four metabolites are used in generating the lipidomic profile, any combination of four of 1-10 listed above can be used. If five metabolites are used in generating the lipidomic profile, any combination of five of 1-10 listed above can be used. If six metabolites are used in generating the lipidomic profile, any combination of six of 1-10 listed above can be used. If seven metabolites are used in generating the lipidomic profile, any combination of seven of 1-10 listed above can be used. If eight metabolites are used in generating the lipidomic profile, any combination of eight of 1-10 listed above can be used. If nine metabolites are used in generating the lipidomic profile, any combination of nine of 1-10 listed above can be used. Of course, all ten metabolites of 1-10 above can be used to generate the lipidomic profile.

Table 1 below lists an exemplary analysis of metabolites 1-10. The normalized ratios depict relative abundance of metabolites in the Cₚᵣₑ group (noted here as Con-pre) as compared to the NC group. Herein, a ratio of one (1) indicates no change while values less than one indicate decreased abundance in the diagnostic group as compared to the NC, or vice versa.

TABLE 1
Metabolite Levels

| | | Normalized Ratio |
|---|---|---|
| Lipid Name | Description | (Con-pre/NC) |
| PC aa C36:6 | Phosphatidylcholine diacyl C36:6 | 0.85 |
| PC aa C38:0 | Phosphatidylcholine diacyl C38:0 | 0.89 |
| PC aa C38:6 | Phosphatidylcholine diacyl C38:6 | 0.86 |
| PC aa C40:1 | Phosphatidylcholine diacyl C40:1 | 0.9 |
| PC aa C40:2 | Phosphatidylcholine diacyl C40:2 | 0.9 |
| PC aa C40:6 | Phosphatidylcholine diacyl C40:6 | 0.77 |
| PC ae C40:6) | Phosphatidylcholine acy-alkyl | 0.89 |
| | C40:6 | |
| LysoPC a C18:2 | Lysophophatidylcholine acyl C18:2 | 0.88 |
| C3 | Propionylacylcarnitine | 0.73 |
| C16:1-OH | Hydroxyhexadecenoylcarnitine | 0.88 |

Techniques to assay levels of individual components of the gene expression profile from test samples are well known to the skilled technician, and the invention is not limited by the means by which the components are assessed. In one embodiment, levels of the individual components of the gene expression profile are assessed using quantitative arrays, PCR, Northern Blot analysis, Western Blot analysis, mass spectroscopy, high-performance liquid chromatography (HPLC) and the like. Other methods of assessing levels of the individual components include biological methods, such as but not limited to ELISA assays. To determine levels of gene expression, it is not necessary that an entire protein or an entire RNA transcript, both of which represent a “gene product,” be present or fully sequenced. In other words, determining levels of, for example, a fragment of an RNA transcript from a gene being analyzed may be sufficient to conclude or assess that the individual gene being analyzed is up- or down-regulated. Similarly, determining levels of, for example, a fragment of a protein encoded by a gene being analyzed may be sufficient to conclude or assess that the individual gene being analyzed is up- or down-regulated. Similarly, if, for example, arrays or blots are used to determine gene expression levels, the presence/absence/strength of a detectable signal will be sufficient to assess levels of gene expression without the need to sequencing an RNA transcript or protein sequence.

The assessment of the levels of the individual components of the gene expression profile can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the individual components of the gene expression profile, a sample is taken from the subject. The sample may or may not processed prior assaying levels of the components of the gene expression profile. For example, whole blood may be taken from an individual and the blood sample may be processed, e.g., centrifuged, to isolate specific cells, e.g., leukocytes, from the blood. The sample may or may not be stored, e.g., frozen, prior to processing or analysis.

Individual components of the gene expression profile include but are not limited to (A) APOBEC3A, (B) ASXL1, (C) CLK4, (D) FAM217B, (E) LYPLA1, (F) OXR1, (G) SCLY, (H) STAG2, and (I) TVP23C-CDRT4. Those of skill in the art will recognize the specific identity of each constituent listed based upon the nomenclature above. For example, gene (E) (LYPLA1) is lysophospholipase 1, gene (H) (STAG2) is stromal antigen 2. The differentially expressed genes are in Table 2 (designated by specific gene symbols (A)-(I)). In one embodiment, the differentially expressed genes are upregulated compared to normal levels. In another embodiment, one, two, three, four, five, six, seven or eight of the genes are upregulated over normal levels.

The levels of upregulation over normal levels can vary. In one embodiment, the levels of gene (A) APOBEC3A are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (B) ASXL1 are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (C) CLK4 are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (D) FAM217B are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (E) LYPLA1 are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (F) OXR1 are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (G) SCLY are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (H) STAG2 are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of gene (I) TVP23C-CDRT4 are upregulated at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. For the purposes of the present invention, the number of “times” the levels of gene expression is lower or higher over normal can be a relative or absolute number of times. In the alternative, the levels of gene expression may be normalized to a standard and these normalized levels can then be compared to one another to determine if gene expression is lower or higher.

TABLE 2
List of Differentially Expressed Genes
for use in the Gene Expression Profile

| Symbol | Entrez Gene Name | Location | Type(s) |
|---|---|---|---|
| (A) APOBEC3A | apolipoprotein B mRNA | Cytoplasm | enzyme |
| | editing enzyme, catalytic | | |
| | polypeptide-like 3A | | |
| (B) ASXL1 | additional sex combs like 1 | Nucleus | transcrip- |
| | (Drosophila) | | tion |
| | | | regulator |
| (C) CLK4 | CDC-like kinase 4 | Nucleus | kinase |
| (D) FAM217B | family with sequence similar- | Other | other |
| | ity 217, member B | | |
| (E) LYPLA1 | lysophospholipase I | Cytoplasm | enzyme |
| (F) OXR1 | oxidation resistance 1 | Cytoplasm | other |
| (G) SCLY | selenocysteine lyase | Cytoplasm | enzyme |
| (H) STAG2 | stromal antigen 2 | Nucleus | other |
| (I) TVP23C- | TVP23C-CDRT4 readthrough | Other | other |
| CDRT4 | | | |

Table 3 lists an exemplary gene expression analysis of the genes listed in Table 1. The log ratios are comparisons of amounts to normal. A positive value indicates an increase (or upregulation) compared to normal levels. A negative value would indicate a decrease (or downregulation) compared to normal levels.

TABLE 3
Log Ratios of Gene Expression

| | | | Log |
|---|---|---|---|
| Symbol | Entrez Gene Name | Location | Ratio |
| (A) APOBEC3A | apolipoprotein B mRNA | Cytoplasm | 8.065 |
| | editing enzyme, catalytic | | |
| | polypeptide-like 3A | | |
| (B) ASXL1 | additional sex combs like 1 | Nucleus | 10.504 |
| | (Drosophila) | | |
| (C) CLK4 | CDC-like kinase 4 | Nucleus | 3.621 |
| (D) FAM217B | family with sequence similar- | Other | 10.530 |
| | ity 217, member B | | |
| (E) LYPLA1 | lysophospholipase I | Cytoplasm | 1.246 |
| (F) OXR1 | oxidation resistance 1 | Cytoplasm | 1.786 |
| (G) SCLY | selenocysteine lyase | Cytoplasm | 10.526 |
| (H) STAG2 | stromal antigen 2 | Nucleus | 12.462 |
| (I) TVP23C- | TVP23C-CDRT4 readthrough | Other | 5.343 |
| CDRT4 | | | |

For the purposes of the present invention the gene expression profile comprises at least two, three, four, five, six, seven, eight or nine genes listed above. If two genes are used in generating the gene expression profile, any combination of two genes of (A)-(I) listed above can be used. If three genes are used in generating the gene expression profile, any combination of three genes of (A)-(I) listed above can be used. If four genes are used in generating the gene expression profile, any combination of four genes of (A)-(I) listed above can be used. If five genes are used in generating the gene expression profile, any combination of five genes of (A)-(l) listed above can be used. If six genes are used in generating the gene expression profile, any combination of six genes of (A)-(I) listed above can be used. If seven genes are used in generating the gene expression profile, any combination of seven genes of (A)-(I) listed above can be used. If eight genes are used in generating the gene expression profile, any combination of eight genes of (A)-(I) listed above can be used. If nine genes are used in generating the gene expression profile, all nine genes of (A)-(I) listed above are to be used.

In one embodiment, the lipidomic profile is assessed prior to determination of the gene expression profile. In this embodiment, the results from the lipidomic profile can be used as a screening tool for further analysis, such as subsequently determining the gene expression profile. For example, the lipidomic profile is assessed for a subject and, based on these initial results, a sample taken from the subject can then be assayed for the gene expression profile to generate the biomarker profile as described herein, which can then be used to determine the subject's likelihood or risk of suffering from memory impairment.

In another embodiment, the gene expression profile is assessed prior to determination of the lipidomic profile. In this embodiment, the results from the gene expression profile can be used as a screening tool for further analysis, such as subsequently determining the lipidomic profile. For example, the gene expression profile is assessed for a subject and, based on these initial results, a sample taken from the subject can then be assayed for the lipidomic profile to generate the biomarker profile as described herein, which can then be used to determine the subject's likelihood or risk of suffering from memory impairment.

In another embodiment, the lipidomic profile and the gene expression profile are assessed contemporaneously and the results are combined to generate the biomarker profile as described herein.

In specific embodiments, all 19 of the markers described herein (10 lipids+9 genes) are combined into one analysis such that there is not a separate lidpidomic profile and a gene expression profile performed. Instead, in this specific embodiment, metabolite levels and gene expression levels are individually assessed and then each individual assessment is compared to its established normal value to determine if each metabolite and expressed gene is at a level that is higher or lower (or not different than) normal value. In these embodiments, at least two, three, four, five, six, seven, eight, nine ten, 11, 12, 13, 14, 15, 16, 17, 18 or 19 markers are used in the analysis.

If two markers are used in the analysis, any combination zero to two of metabolites 1-10 and/or zero to two of genes (A)-(I) listed herein above can be used. If three markers are used in the analysis, any combination zero to three of metabolites 1-10 and/or zero to three of genes (A)-(I) listed herein above can be used. If four markers are used in the analysis, any combination zero to four of metabolites 1-10 and/or zero to four of genes (A)-(I) listed herein above can be used. If five markers are used in the analysis, any combination zero to five of metabolites 1-10 and/or zero to five of genes (A)-(I) listed herein above can be used. If six markers are used in the analysis, any combination zero to six of metabolites 1-10 and/or zero to six of genes (A)-(I) listed herein above can be used. If seven markers are used in the analysis, any combination zero to seven of metabolites 1-10 and/or zero to seven of genes (A)-(I) listed herein above can be used. If eight markers are used in the analysis, any combination zero to eight of metabolites 1-10 and/or zero to eight of genes (A)-(I) listed herein above can be used. If nine markers are used in the analysis, any combination zero to nine of metabolites 1-10 and/or zero to nine of genes (A)-(I) listed herein above can be used. If ten markers are used in the analysis, any combination one to ten of metabolites 1-10 and/or zero to nine of genes (A)-(I) listed herein above can be used. If 11 markers are used in the analysis, any combination two to ten of metabolites 1-10 and/or one to nine of genes (A)-(I) listed herein above can be used. If 12 markers are used in the analysis, any combination three to ten of metabolites 1-10 and/or two to nine of genes (A)-(I) listed herein above can be used. If 13 markers are used in the analysis, any combination four to ten of metabolites 1-10 and/or three to nine of genes (A)-(I) listed herein above can be used. If 14 markers are used in the analysis, any combination five to ten of metabolites 1-10 and/or four to nine of genes (A)-(I) listed herein above can be used. If 15 markers are used in the analysis, any combination six to ten of metabolites 1-10 and/or five to nine of genes (A)-(I) listed herein above can be used. If 16 markers are used in the analysis, any combination seven to ten of metabolites 1-10 and/or six to nine of genes (A)-(I) listed herein above can be used. If 17 markers are used in the analysis, any combination eight to ten of metabolites 1-10 and/or seven to nine of genes (A)-(l) listed herein above can be used. If 18 markers are used in the analysis, any combination nine to ten of metabolites 1-10 and/or eight to nine of genes (A)-(I) listed herein above can be used. If all 19 markers are used in the analysis, then all of metabolites 1-10 and all of genes (A)-(I) listed herein are be used.

The subject's biomarker profile (lipidomic profile and gene expression profile) is compared to the profile that is deemed to be a normal biomarker profile (lipidomic profile and gene expression profile). To establish the biomarker profile (lipidomic profile and gene expression profile) of a normal individual, an individual or group of individuals may be first assessed for their ability to recall past events to establish that the individual or group of individuals has a normal or acceptable ability memory. Once established, the biomarker profile (lipidomic profile and gene expression profile) of the individual or group of individuals can then be determined to establish a “normal biomarker profile” (“normal lipidomic profile” and “normal gene expression profile”). In one embodiment, a normal biomarker profile (lipidomic profile and gene expression profile) can be ascertained from the same subject when the subject is deemed to possess normal cognitive abilities and no signs (clinical or otherwise) of memory impairment. In one embodiment, a “normal” biomarker profile (lipidomic profile and gene expression profile) is assessed in the same subject from whom the sample is taken prior to the onset of measureable, perceivable or diagnosed memory impairment. That is, the term “normal” with respect to a biomarker profile (lipidomic profile and gene expression profile) can be used to mean the subject's baseline biomarker profile (lipidomic profile and gene expression profile) prior to the onset of memory impairment. The biomarker profile (lipidomic profile and gene expression profile) can then be reassessed periodically and compared to the subject's baseline biomarker profile (lipidomic profile and gene expression profile). Thus, the present invention also include methods of monitoring the progression of memory impairment in a subject, with the methods comprising determining the subject's biomarker profile (lipidomic profile and gene expression profile) more than once over a period of time. For example, some embodiments of the methods of the present invention will comprise determining the subject's biomarker profile (lipidomic profile and gene expression profile) two, three, four, five, six, seven, eight, nine, 10 or even more times over a period of time, such as a year, two years, three, years, four years, five years, six years, seven years, eight years, nine years or even 10 years or longer. The methods of monitoring a subject's risk of having memory impairment would also include embodiments in which the subject's biomarker profile (lipidomic profile and gene expression profile) is assessed during and after treatment of memory impairment. In other words, the present invention also includes methods of monitoring the efficacy of treatment of memory impairment by assessing the subject's biomarker profile (lipidomic profile and gene expression profile) over the course of the treatment and after the treatment. The treatment may be any treatment designed to increase a subject's ability to recall past events, i.e., improve a subject's memory.

In another embodiment, a normal biomarker profile (lipidomic profile and gene expression profile) is assessed in a sample from a different subject or patient (from the subject being analyzed) and this different subject does not have or is not suspected of having memory impairment. In still another embodiment, the normal biomarker profile (lipidomic profile and gene expression profile) is assessed in a population of healthy individuals, the constituents of which display no memory impairment. Thus, the subject's biomarker profile (lipidomic profile and gene expression profile) can be compared to a normal biomarker profile (lipidomic profile and gene expression profile) generated from a single normal sample or a biomarker profile (lipidomic profile and gene expression profile) generated from more than one normal sample.

Of course, measurements of the individual components, e.g., concentration, of the normal biomarker profile (lipidomic profile and gene expression profile) can fall within a range of values, and values that do not fall within this “normal range” are said to be outside the normal range. These measurements may or may not be converted to a value, number, factor or score as compared to measurements in the “normal range.” For example, a measurement for a specific metabolite that is below the normal range, may be assigned a value or −1, −2, −3, etc., depending on the scoring system devised.

In one embodiment, the “biomarker profile value” can be a single value, number, factor or score given as an overall collective value to the individual molecular components of the profile, or to the categorical components, i.e., the lipidomic profile and the gene expression profile. For example, if each component is assigned a value, such as above, the biomarker value may simply be the overall score of each individual or categorical value. For example, if 10 components are used to generate the lipidomic profile and five of the components are assigned values of “−2” and five are assigned values of “−1,” the lipidomic profile portion of the biomarker profile value in this example would be −15, with a normal value being, for example, “0.” Continuing the example, if 9 components are used to generate the gene expression profile and five of the components are assigned values of “2” and four are assigned values of “−1,” the gene expression profile portion of the biomarker profile value in this example would be 6, with a normal value being, for example “0.” In this manner, the biomarker profile value could be useful single number or score, the actual value or magnitude of which could be an indication of the actual risk of memory impairment, e.g., the “more negative” the value, the greater the risk of memory impairment.

In another embodiment the “biomarker profile value” can be a series of values, numbers, factors or scores given to the individual components of the overall profile. In another embodiment, the “biomarker profile value” may be a combination of values, numbers, factors or scores given to individual components of the profile as well as values, numbers, factors or scores collectively given to a group of components. For example, the measurements of the phosphatidylcholines in the profile may be grouped into one composite score, individual acylcarnitines may be grouped into another composite score and differential expression of enzymes may be grouped into another score. In another example, the biomarker profile value may comprise or consist of individual values, number, factors or scores for specific components, e.g., metabolite 3 (PC aa C36:6), as well as values, numbers, factors or scores for a group on components.

In another embodiment individual biomarker values from the metabolites and genes can be used to develop a single score, such as a “combined biomarker index,” which may utilize weighted scores from the individual biomarker values reduced to a diagnostic number value. The combined biomarker index may also be generated using non-weighted scores from the individual biomarker values from the metabolites and genes. When the “combined biomarker index” exceeds (or is less than) a specific threshold level, the individual has a high risk of memory impairment, whereas the maintaining a normal range value of the “combined biomarker index” would indicate a low or minimal risk of memory impairment. In this embodiment, the threshold value would be set by the combined biomarker index from normal subjects.

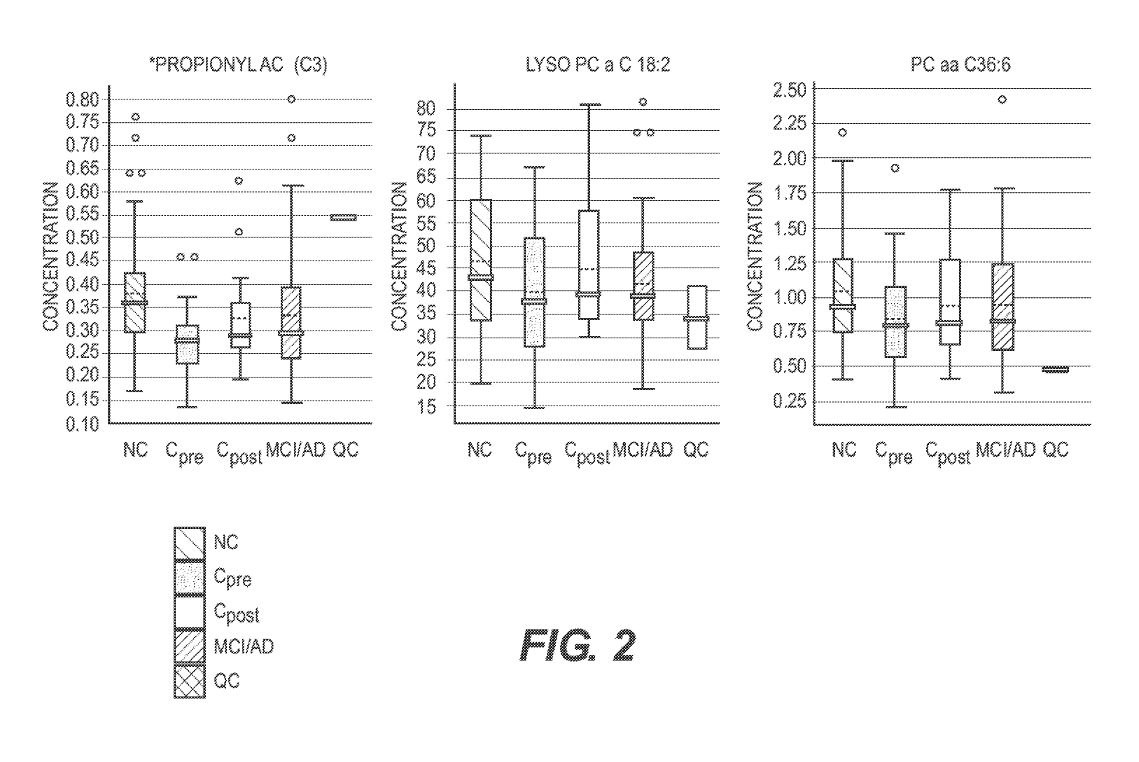

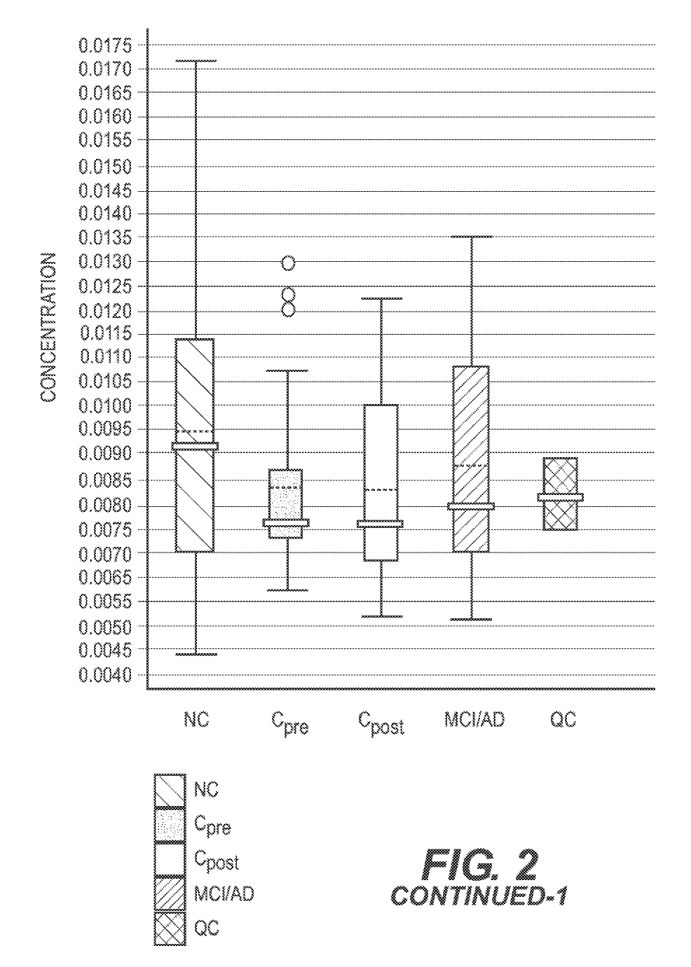

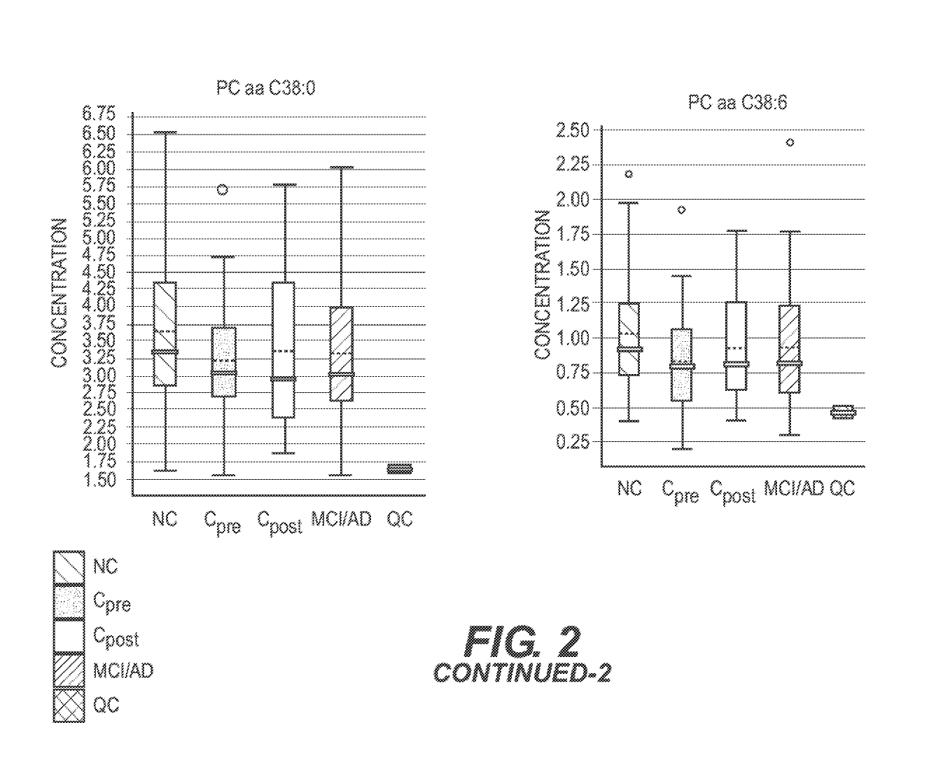

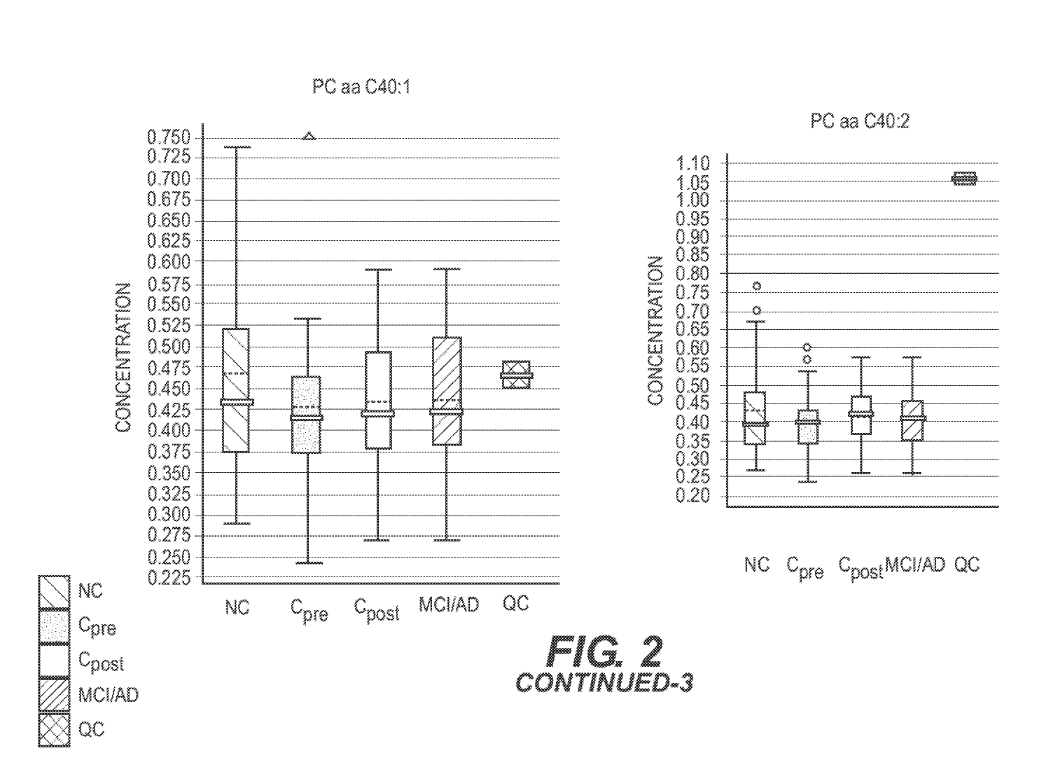

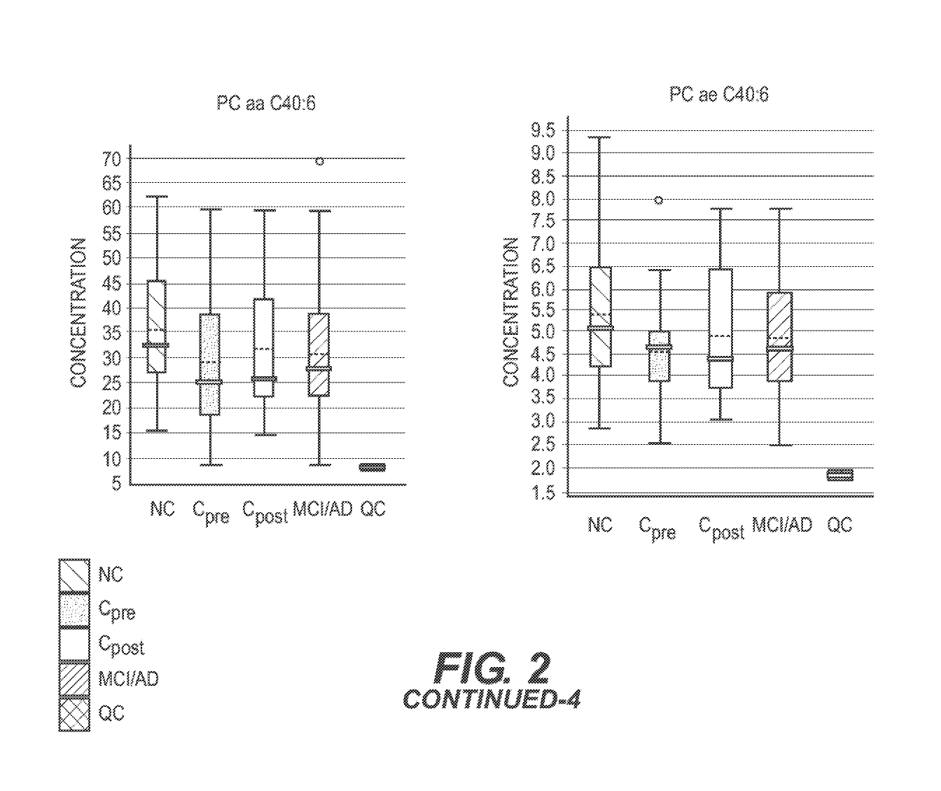

In another embodiment, the value of the biomarker profile can be the collection of data from the individual measurements and need not be converted to a scoring system, such that the “biomarker profile value” is a collection of the individual measurements of the individual components of the profile. For example, the value of the lipidomic component of the biomarker profile may be a collection of measurements as seen in FIG. 2.

In specific embodiments, a subject is diagnosed of having an increased risk of suffering from memory impairment if the subject's 19 of the markers described herein (10 lipids+9 genes) are at abnormal levels, e.g., all of the lipid metabolites are lower than normal levels and all of genes are expressed at higher levels. In other specific embodiments, a subject is diagnosed of having an increased risk of suffering from memory impairment if 18 the subject's 19 markers described herein (10 lipids+9 genes) are at abnormal levels, e.g., all or all but one of the lipid metabolites are lower than normal levels and all or all but one of the genes are expressed at higher levels. In other specific embodiments, a subject is diagnosed of having an increased risk of suffering from memory impairment if 17 the subject's 19 markers described herein (10 lipids+9 genes) are at abnormal levels, e.g., anywhere from zero to two of the lipid metabolites are not lower than normal levels and anywhere from zero to two of the genes are not expressed at higher levels. In other specific embodiments, a subject is diagnosed of having an increased risk of suffering from memory impairment if 16 the subject's 19 markers described herein (10 lipids+9 genes) are at abnormal levels, e.g., anywhere from zero to nine of the lipid metabolites are not lower than normal levels and anywhere from zero to nine of the genes are not expressed at higher levels. In other specific embodiments, a subject is diagnosed of having an increased risk of suffering from memory impairment if 15 the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if 14 the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if 13 the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if 12 the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if 11 the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if ten the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if nine the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if eight the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if seven the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if six the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if five the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if four the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if three the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels. In other specific embodiments, a subject is diagnosed of having an increased risk of suffering from memory impairment if two the subject's 19 markers described herein (10 lipids+9 genes) are at abnormal levels, e.g., anywhere from zero to ten of the lipid metabolites are not lower than normal levels and anywhere from zero to nine of the genes are not expressed at higher levels.

If it is determined that a subject has an increased risk of memory impairment, the attending health care provider may subsequently prescribe or institute a treatment program. In this manner, the present invention also provides for methods of screening individuals as candidates for treatment of memory impairment. The attending healthcare worker may begin treatment, based on the subject's biomarker profile, before there are perceivable, noticeable or measurable signs of memory impairment in the individual.

Similarly, the invention provides methods of monitoring the effectiveness of a treatment for memory impairment. Once a treatment regimen has been established, with or without the use of the methods of the present invention to assist in a diagnosis of memory impairment, the methods of monitoring a subject's biomarker profile over time can be used to assess the effectiveness of a memory impairment treatment. Specifically, the subject's biomarker profile can be assessed over time, including before, during and after treatments for memory impairment. The biomarker profile can be monitored, with, for example, a decline in the values of the profile over time being indicative that the treatment may not be as effective as desired.

All patents and publications mentioned in this specification are indicative of the level of those skilled in the art to which the invention pertains. All patents and publications cited herein are incorporated by reference to the same extent as if each individual publication was specifically and individually indicated as having been incorporated by reference in its entirety

### EXAMPLES

### Example 1

### Neurocognitive Methods

A total of 525 volunteers participated in this study as part of the Rochester/Orange County Aging Study (R/OCAS), an ongoing natural history study of cognition in community-dwelling older adults. Briefly, participants were followed with yearly cognitive assessments and blood samples were collected following an overnight fast and withholding of all medications. At baseline and each yearly visit, participants completed assessments in such as activities in daily living, memory complaints, signs and symptoms of depression, and were administered a detailed cognitive assessment.

For this study, data from the cognitive tests were used to classify participants into groups for biomarker discovery. Standardized scores (Z-scores) were derived for each participant on each cognitive test and the composite Z-scores were computed for five cognitive domains (attention, executive, language, memory, visuoperceptual) (Table 4).

TABLE 4

| | | Lan- | Visuoper- | |
|---|---|---|---|---|
| Attention | Executive | guage | ceptual | Memory |
| (Zₐₜₜ) | (Zₑₓₑ) | (Zₗₐₙ) | (Zᵥᵢₛ) | (Zₘₑₘ) |
| Wechsler | Wechsler | 1-min | Hooper | Rey Auditory |
| Memory | Memory | Cate- | Visual | Verbal Learning |
| Scale-III | Scale-III | gory | Organiza- | Test Learning |
| Forward | Backward | fluency | tion Test | (RAVLT |
| Digit Span | Digit Span | (Ani- | (HVOT) | Learning) |
| (WMS-III | (WMS-III | mals) | | |
| FDS) | BDS) | | | |
| Trail Making | Trail Making | Boston | | Rey Auditory |
| Test- Part A | Test- Part B | Naming | | Verbal Learning |
| (TMT-A) | (TMT-B) | Test 60- | | Test Retrieval |
| | | Item | | (RAVLT |
| | | version | | Retrieval) |
| | | (BNT-60) | | |
| | | | | Rey Auditory |
| | | | | Verbal Learning |
| | | | | Test Retention |
| | | | | (RAVLT |
| | | | | Recognition) |

Normative data for Z-score calculations were derived from the performance of the participants on each of the cognitive tests adjusted for age, education, sex, and visit. To reduce the effect of cognitively impaired participants on the mean and SD, age-, education-, sex, and visit-adjusted residuals from each domain Z-score model were robustly standardized to have median 0 and robust SD=1, where the robust SD=IQR/1.35, as 1.35 is the IQR (Inter-Quartile Range) of a standard normal distribution.

The participants were then categorized into groups of incident aMCI or early AD (combined into one category aMCI/AD), cognitively normal control (NC), and those who converted to MCI or AD over the course of the study (Converters) based on these composite scores. Impairment was defined as a Z-score 1.35 SD below the cohort median. All participants classified as aMCI met recently revised criteria for the amnestic subtype of MCI. Other behavioral phenotypes of MCI were excluded to concentrate on the amnestic form, which most likely represents nascent Alzheimer's pathology. All early AD participants met recently revised criteria for probable Alzheimer's disease with impairment in memory and at least one other cognitive domain. For the MCI and early AD groups, scores on the measures of memory complaints (MMQ) and activities of daily living (PGC-IADL) were used to corroborate research definitions of these states. All Converters had non-impaired memory at entry to the study (Zₘₑₘ≧−1.35), developed memory impairment over the course of the study (Zₘₑₘ≦−1.35) and met criteria for the above definitions of aMCI or AD. To enhance the specificity of the biomarker analyses, NC participants in this study were conservatively defined with Zₘₑₘ±1 SD of the cohort median rather than simply ≧−1.35, and all other Z-scores ≧−1.35 SD.

For each subject, Zₘₑₘ(last), Zₐₜₜ(last), Zₑₓₑ(last), Zₗₐₙ(last), and Zᵥᵢₛ(last) were defined as the age-gender-education-visit-adjusted robust Z-scores for the last available visit for each subject. The aMCI/AD group was defined as those participants whose adjusted Zₘₑₘ was 1 IQR below the median at their last available visit, i.e., Zₘₑₘ(last)≦−1.35. Converters were defined as that subset of the aMCI/AD group whose adjusted Zₘₑₘ at baseline visit 0 was no more than 1 IQR below the median, i.e., Zₘₑₘ(visit=0)>−1.35 and Zₘₑₘ(last)≦−1.35. Participants were classified as NC if they had central scores on all domains at both the first and last visits, i.e., only if they met all of the following six conditions: (i) −1<Zₘₑₘ(last)<1, (ii) −1<Zₘₑₘ(visit=0)<1, (iii) Zₘᵢₙ(last)>−1.35, (iv) Zₘᵢₙ(visit=0) >−1.35, (v) Zₘₐₓ(last)<1.35, and (vi) Zₘₐₓ(visit=0)<1.35, where Zₘₐₓ(last) and Zₘₐₓ(visit=0) denote the maximum of the five adjusted Z-scores at the last and first visits, respectively. Zₘₑₘ for normal participants had to be within 0.74 IQR (1 SD) of the median, rather than just 1 IQR (1.35 SD), to guarantee that they were >0.25 IQR (0.35 SD) from aMCI/AD participants.

After three years of being in the study, (December, 2010), 202 participants had completed a baseline and two yearly visits. At the third visit, 53 participants met criteria for aMCI/AD and 96 met criteria for NC. Of the 53 aMCI/AD participants, 18 were Converters and 35 were incident aMCI or AD. The remaining 53 participants did not meet the criteria for either group and were not considered for biomarker profiling. Some of these individuals met criteria for non-amnestic MCI and many had borderline or even above average memory scores that precluded their inclusion as either aMCI/AD or NC. 53 of the NC participants were matched to the 53 aMCI/AD participants based on sex, age, and education level. Blood samples were obtained on the last available study visit for the 53 MCI/AD and the 53 NC for biomarker discovery. Two blood samples from each of the 18 Converters were also included: one from the baseline visit (Cₚᵣₑ) when Zₘₑₘ was non-impaired and one from the third visit (Cₚₒₛₜ) when Zₘₑₘ was impaired and they met criteria for either aMCI or AD. Thus, at total of 124 samples from 106 participants were analyzed.

Internal cross-validation was employed to validate findings from the discovery phase. Blood samples for validation were identified at the end of the fifth year of the study and all 106 participants included in the discovery phase were excluded from consideration for the validation phase. Cognitive composite Z-scores were re-calculated based on the entire sample available and the same procedure and criteria were used to identify samples for the validation phase. A total of 145 participants met criteria for a group: 21aMCI/AD and 124 NC. Of the 21 aMCI/AD, 10 were Converters. 20 of the NC participants were matched to the aMCI/AD participants on the basis of age, sex, and education level as in the discovery phase. In total, 41 participants contributed samples to the validation phase and, as before, the 10 Converters also contributed a baseline sample (Cₚᵣₑ) for a total of 51 samples.

Neurocognitive Statistical Analyses

The neurocognitive analyses were designed to demonstrate the general equivalence of the discovery and validation samples on clinical and cognitive measures. Separate Multivariate Analysis of Variance (MANOVA's) tests were used to examine discovery/validation group performance on the composite Z-scores and on self-report measures of memory complaints, memory related functional impairment, depressive symptoms, and a global measure of cognitive function. In the first MANOVA, biomarker sample (discovery, validation) was the independent variable and MMQ, IADL, GDS, and MMSE were the dependent variables. In the second MANOVA, biomarker sample (discovery, validation) was the independent variable and the five cognitive domain Z-scores (Zₐₜₜ, Zₑₓₑ, Zₗₐₙ, Zₘₑₘ, and Zᵥᵢₛ) were the dependent variables. Significance was set at alpha=0.05 and Tukey's HSD procedure was used for post-hoc comparisons. All statistical analyses were performed using SPSS (version 21).

### Example 2

### Lipidomics Reagents

LC/MS-grade acetonitrile (ACN), Isopropanol (IPA), water and methanol were purchased from Fisher Scientific (New Jersey, USA). High purity formic acid (99%) was purchased from Thermo-Scientific (Rockford, Ill.). Debrisoquine, 4-Nitrobenzoic acid (4-NBA), Pro-Asn, Glycoursodeoxycholic acid, Malic acid, were purchased from Sigma (St. Louis, Mo., USA). All lipid standards including 14:0 LPA, 17:0 Ceramide, 12:0 LPC, 18:0 Lyso PI and PC(22:6/0:0) were procured from Avanti Polar Lipids Inc. (USA).

Metabolite Extraction

Briefly, the plasma samples were thawed on ice and vortexed. For metabolite extraction, 25 μL of plasma sample was mixed with 175 μL of extraction buffer (25% acetonitrile in 40% methanol and 35% water) containing internal standards [10 μL of debrisoquine (1 mg/mL), 50A of 4, nitro-benzoic acid (1 mg/mL), 27.3 μl of Ceramide (1 mg/mL) and 2.5 μL of LPA (lysophosphatidic acid) (4 mg/mL) in 10 mL). The samples were incubated on ice for 10 minutes and centrifuged at 14,000 rpm at 4° C. for 20 minutes. The supernatant was transferred to a fresh tube and dried under vacuum. The dried samples were reconstituted in 200 μL of buffer containing 5% methanol, 1% acetonitrile and 94% water. The samples were centrifuged at 13,000 rpm for 20 minutes at 4° C. to remove fine particulates. The supernatant was transferred to a glass vial for UPLC-ESI-Q-TOF-MS analysis.

UPLC-ESI-QTOF-MS Based Data Acquisition for Untargeted Lipidomic Profiling

Each sample (24) was injected onto a reverse-phase CSH C18 1.7 μM 2.1×100 mm column using an Acquity H-class UPLC system (Waters Corporation, USA). The gradient mobile phase comprised of water containing 0.1% formic acid solution (Solvent A), 100% acetonitrile (Solvent B) and 10% acetonitrile in isopropanol (IPA) containing 0.1% formic acid and 10 mM ammonium formate (Solvent C). Each sample was resolved for 13 minutes at a flow rate of 0.5 mL/min for 8 min and then 0.4 mL/min from 8 to 13 min. The UPLC gradient consisted of 98% A and 2% B for 0.5 min then a ramp of curve 6 to 60% B and 40% A from 0.5 min to 4.0 min, followed by a ramp of curve 6 to 98% B and 2% A from 4.0 to 8.0 min, then ramped to 5% B and 95% C from 9.0 min to 10.0 min at a flow rate of 0.4 ml/min, and finally to 98% A and 2% B from 11.0 min to 13 minutes. The column eluent was introduced directly into the mass spectrometer by electrospray ionization. Mass spectrometry was performed on a Quadrupole-Time of Flight (Q-TOF) instrument (Xevo G2 QTOF, Waters Corporation, USA) operating in either negative (ESI⁻) or positive (ESI⁺) electrospray ionization mode with a capillary voltage of 3200 V in positive mode and 2800 V in negative mode, and a sampling cone voltage of 30 V in both modes. The desolvation gas flow was set to 750 L h⁻¹ and the temperature was set to 350° C. while the source temperature was set at 120° C. Accurate mass was maintained by introduction of a lock spray interface of leucineenkephalin (556.2771 [M+H] or 554.2615 [M−H]) at a concentration of 2 pg/μl in 50% aqueous acetonitrile and a rate of 2 μl/min. Data were acquired in centroid MS mode from 50 to 1200 m/z mass range for TOE-MS scanning as single injection per sample and the batch acquisition was repeated to check experimental reproducibility. For the metabolomics profiling experiments, pooled quality control (QC) samples (generated by taking an equal aliquot of all the samples included in the experiment) were run at the beginning of the sample queue for column conditioning and every ten injections thereafter to assess inconsistencies that are particularly evident in large batch acquisitions in terms of retention time drifts and variation in ion intensity over time. This approach has been recommended and used as a standard practice by leading meta bolomics researchers. A test mix of standard metabolites was run at the beginning and at the end of the run to evaluate instrument performance with respect to sensitivity and mass accuracy. The sample queue was randomized to remove bias.

Stable Isotope—Dilution Multiple Reaction Monitoring Mass Spectrometry (SID-MRM-MS)

Targeted metabolomic analysis of plasma sample was performed using the Biocrates Absolute-IDQ P180 (BIOCRATES, Life Science AG, Innsbruck, Austria). This validated targeted assay allows for simultaneous detection and quantification of metabolites in plasma samples (10 μL) in a high throughput manner. The methods have been described in detail. The plasma samples were processed as per the instructions by the manufacturer and analyzed on a triple quadrupole mass spectrometer (Xevo TQ-S, Waters Corporation, USA) operating in the MRM mode. The measurements were made in a 96 well format for a total of 148 samples, seven calibration standards and three quality control samples were integrated in the kit.

Briefly, the flow injection analysis (FIA) tandem mass spectrometry (MS/MS) method was used to quantify a panel of 144 lipids simultaneously by multiple reaction monitoring. The other metabolites were resolved on the UPLC and quantified using scheduled MRMs. The kit facilitated absolute quantitation of 21 amino acids, hexose, carnitine, 39 acylcarnitines, 15 sphingomyelins, 90 phosphatidylcholines and 19 biogenic amines. Data pre-processing was performed using the MetIQ software (Biocrates) while the statistical analyses were performed using linear regression as well as the STAT pack module v3 (Biocrates). The concentration is expressed as nmol/L. Quality control samples were used to assess reproducibility of the assay. The mean of the coefficient of variation (CV) for the 180 metabolites was 0.08 and 95% of the metabolites had a CV of <0.15.

Lipidomics Statistical Analyses

The rn/z features of metabolites were normalized with log transformation that stabilized the variance followed with a quantile normalization to make the empirical distribution of intensities the same across samples. The metabolites were selected among all those known to be identifiable using a ROC regularized learning technique, based on the least absolute shrinkage and selection operator (LASSO) penalty as implemented with the R package ‘glmnet’, which uses cyclical coordinate descent in a pathwise fashion. The regularization path over a grid of values was obtained for the tuning parameter lambda through 10-fold cross-validation. The optimal value of the tuning parameter lambda, which was obtained by the cross-validation procedure, was then used to fit the model. All the features with non-zero coefficients were retained for subsequent analysis. The classification performance of the selected metabolites was assessed using area under the ROC (receiver operating characteristic) curve (AUC). The ROC can be understood as a plot of the probability of classifying correctly the positive samples against the rate of incorrectly classifying true negative samples. Thus the AUC measure of an ROC plot is actually a measure of predictive accuracy. To maintain rigor of independent validation, the simple logistic model with the ten metabolite panel was used, although a more refined model can yield greater AUC.

Results

Over the course of the study, 74 participants met criteria for either aMCI or mild AD, 46 of these were incidental cases at entry and 28 phenoconverted (Converters) from a non-impaired memory status at entry (Cₚᵣₑ). The average time to phenoconversion was 2.1 years (range=1-5 years). 53 aMCI/AD participants were selected, including 18 Converters, and 53 age-, education-, and sex-matched cognitively normal control (NC) participants for untargeted lipidomics biomarker discovery. Internal cross validation was used to evaluate the accuracy of the discovered lipidomics profile in classifying a blinded sample of 51 subjects consisting of the remaining subset of 21 aMCI/AD participants, including 10 Converters, and an additional 20 matched NC.

The aMCI/AD, Converter, and NC groups were defined primarily using a composite measure of memory performance in addition to composite measures of other cognitive abilities and clinical measures of memory complaints and functional capacities. (See Tables 4 and 5).

TABLE 5

| | Dependent | |
|---|---|---|
| | Measure | Domain |
| Clinical/Cognitive Measures | (Range) | Assessed |
| Multiple Assessment Inventory IADL Scale (MAI-IADL) | Total Score | Functional |
| Lawton M P. (1988) Instrumental Activities of Daily Living (IADL) | (0-27) | capacities |
| scale: Original observer-rated version. Psychopharmacology | | |
| Bulletin, 24, 785-7. | | |
| Multifactorial Memory Questionnaire (MMQ) | Total Score | Memory |
| Troyer A K and Rich J B. (2002). Psychometric properties of a new | (0-228) | complaints |
| metamemory questionnaire for older adults. Journal of | | |
| Gerontology, 57(1), 19-27. | | |
| Mini Mental State Examination (MMSE) | Total Score | Global |
| Folstein, M F, Folstein, S E, and McHugh, P R. (1975). “Mini-mental | (0-30) | cognitive |
| state”. Journal of Psychiatric Research, 12, 189-98. | | ability |
| Geriatric Depression Scale-Short Form (GDS-SF) | Total Score | Mood |
| Sheikh J I and Yesavage J A. (1986). Geriatric Depression Scale (GDS): | (0-15) | |
| Recent evidence and development of a shorter version. Clinical | | |
| Gerontologist, 5, 165-173. | | |
| Wechsler Memory Scale-III Forward Digit Span (WMS-III FDS) | Span Length | Attention |
| Wechsler D. Wechsler Memory Scale-III Manual. San Antonio, TX: | (0-9) | |
| The Psychological Corporation, 1997. | | |
| Trail Making Test- Part A (TMT-A) | Completion time | Attention |
| Reitan R M. (1958). Validity of the Trail Making Test as an indicator | (1-300 seconds) | |
| of organic brain damage. Perceptual and Motor Skills, 8, 271-6. | | |
| Wechsler Memory Scale-III Backward Digit Span (WMS-III BDS) | Span Length | Executive |
| Wechsler D. Wechsler Memory Scale-III Manual. San Antonio, TX: | (0-8) | ability |
| The Psychological Corporation, 1997. | | |
| Trail Making Test- Part B (TMT-B) | Completion Time | Executive |
| Reitan R M. (1958). Validity of the Trail Making Test as an indicator | (1-300 seconds) | ability |
| of organic brain damage. Perceptual and Motor Skills, 8, 271-6. | | |
| Category fluency (Animals) | Animals named in | Language |
| Borkowski J, Benton A, Spreen O. (1967). Word fluency and brain | 1-minute | |
| damage. Neuropsychologia, 5, 135-140 | | |
| Boston Naming Test 60-Item version (BNT-60) | Total Correct | Language |
| Kaplan E, Goodglass H, and Weintraub S. (1983). Boston Naming | (0-60) | |
| Test. Philadelphia: Lea & Feibiger. | | |
| Rey Auditory Verbal Learning Test Learning (RAVLT Learning) | Total words | Verbal |
| Rey A. (1964). L'examen clinique en psychologie. Paris: Presses | recalled over | learning |
| Universitaires de France. | Trials 1-5 (0-75) | |
| Rey Auditory Verbal Learning Test Recall (RAVLT Retrieval) | Words recalled at | Verbal |
| Rey A. (1964). L'examen clinique en psychologie. Paris: Presses | 20-minute delay | retrieval |
| Universitaires de France. | (0-15) | |
| Rey Auditory Verbal Learning Test Retention (RAVLT | True positives- | Verbal |
| Recognition) | false positives | retention |
| Rey A. (1964). L'examen clinique en psychologie. Paris: Presses | (0-15) | |
| Universitaires de France. | | |
| Hooper Visual Organization Test (HVOT) | Total score | Visuoper- |
| Hooper H E. Hooper Visual Organization Test (VOT) Los Angeles: | (0-30) | ception |
| Western Psychological Services; 1983. | | |

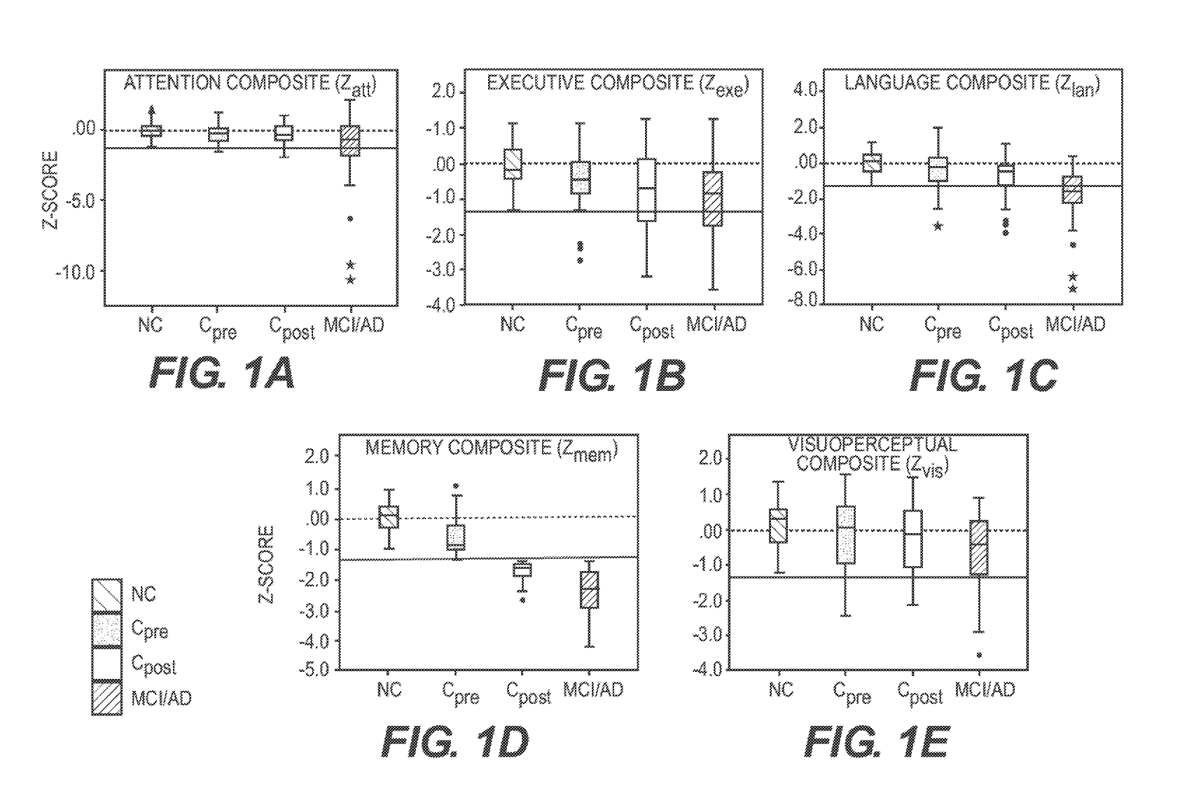

Plots of group means on the composite measures can be found in FIG. 1 and group means are reported in Table 6. The discovery and validation groups did not differ on clinical measures including self-reported memory complaints, functional impairment, depressive symptoms, and a global measure of cognition (F(4,170)=1.376, p=0.244), nor on any composite Z-score (F(5,169)=2.118, p=0.066) demonstrating the general equivalence of the participants used for the discovery and validation phases of the biomarker analysis.

TABLE 6

| | NC | Cₚᵣₑ | MCI/AD |
|---|---|---|---|
| Cognitive Measure | (n = 73) | (n = 28) | (n = 74) |
| Multiple Assessment Inventory IADL | 26.51 | 26.65 | 24.82 |
| Scale (MAI-IADL) | (1.71) | (0.87) | (3.60) |
| Multifactorial Memory Questionnaire | 130.32 | 139.71 | 121.01 |
| (MMQ) | (19.93) | (13.36) | (18.14) |
| Mini Mental State Examination (MMSE) | 28.64 | 28.61 | 26.32 |
| | (1.30) | (2.49) | (2.87) |
| Geriatric Depression Scale-Short Form | 1.47 | 1.32 | 1.97 |
| (GDS-SF) | (2.02) | (2.28) | (2.7) |
| Wechsler Memory Scale-III Forward | 6.25 | 6.18 | 6.14 |
| Digit Span (WMS-III FDS) | (1.05) | (0.95) | (1.13) |
| Trail Making Test- Part A | 36.69 | 46.14 | 55.26 |
| (TMT-A) | (13.23) | (14.75) | (44.63) |
| Wechsler Memory Scale-III Backward | 4.34 | 4.29 | 4.01 |
| Digit Span (WMS-III BDS) | (0.9) | (0.76) | (0.91) |
| Trail Making Test- Part B (TMT-B) | 98.53 | 134.57 | 151.99 |
| | (41.30) | (63.89) | (69.82) |
| Category fluency (Animals) | 20.91 | 19.0 | 15.16 |
| | (4.72) | (5.24) | (5.03) |
| Boston Naming Test 60-Item version | 56.29 | 53.14 | 50.51 |
| (BNT-60) | (3.19) | (7.96) | (9.46) |
| Rey Auditory Verbal Learning Test | 43.43 | 37.0 | 27..08 |
| Learning (RAVLT Total Learning A1-A5) | (7.76) | (5.88) | (7.01) |
| Rey Auditory Verbal Learning Test | 7.84 | 5.32 | 1.93 |
| Delayed Recall (RAVLT Trial A7) | (2.48) | (2.59) | (1.64) |
| Rey Auditory Verbal Learning Test | 13.30 | 11.14 | 7.09 |
| Retention (RAVLT Recognition) | (1.57) | (2.24) | (3.15) |
| Hooper Visual Organization Test | 23.96 | 22.36 | 20.93 |
| (HVOT) | (3.05) | (3.72) | (4.51) |

Lipidomic Definition of Participant Groups

The plasma samples from the 124 discovery phase participants were subjected to lipidomics analysis. In the discovery phase, metabolomic/lipidomic profiling yielded 2700 features in the positive mode and 1900 features in the negative mode. The metabolites that define the participant groups were selected from among all known to be identifiable using a regularized learning technique, the least absolute shrinkage and selection operator (LASSO) penalty as implemented with the R package ‘glmnet’. The LASSO analysis revealed features that assisted in unambiguous class separation between aMCI/AD, Converterₚᵣₑ and the NC group (Table 7).

TABLE 7
Putative metabolite markers resulting from
binary comparison of the study groups

| | Lasso | Comparison | | Mass/Charge |
|---|---|---|---|---|
| Metabolite | Coefficient | Groups | Mode | Ratio |
| PI(18:0/0:0) | ↓ (−0.674) | NC vs Cₚᵣₑ | NEG | 599.3226 |
| Pro Asn | ↑ (0.192) | NC vs Cₚᵣₑ | POS | 230.1146 |
| Glycoursodeoxy- | ↑ (0.107) | NC vs Cₚᵣₑ | POS | 450.3196 |
| cholic acid | | | | |
| Malic acid | ↓ (−0.024) | NC vs aMCI/AD | POS | 134.0207 |

The markers in Table 7 were chosen based on the significant predictive value as determined by LASSO coefficient analysis. The positive estimated LASSO coefficient suggests elevation in corresponding comparison group (aMCI/AD and Cₚᵣₑ) compared to normal control (NC) participants. Up arrows indicate up-regulation in the comparison group as compared to the NC participants while the down arrow suggests down-regulation in these groups.

This untargeted lipidomic analysis revealed a significant decrease in the level of phosphatidyl inositol (PI) in the Cₚᵣₑ group and an elevation in the plasma levels of glycoursodeoxycholic acid in the aMCI/AD patients as compared to the NC group. These metabolites were unambiguously identified using tandem mass spectrometry. The other metabolites that displayed differential abundance in the study groups consisted of amino acids, biogenic amines and a broad range of phospholipids and other lipid species that were putative identified based on accurate mass stringency of 5 ppm.

In the next step of the metabolomic/lipidomic analyses, multiple reaction monitoring (MRM) was performed for stable isotope dilution—mass spectrometry (SID-MS) to unambiguously identify and quantify those metabolites such as lipids, amino acids and biogenic amines that function similar to those identified in the LASSO analysis and characterize the participant groups, with special emphasis on differences that would predict a predisposition of phenoconversion from NC to aMCI/AD. The data revealed significantly lower plasma levels of serotonin, taurine, phenylalanine, proline, lysine, phosphatidyl choline (PC) and acylcarnitine (AC) in Cₚᵣₑ participants who later developed aMCI/AD. Conversely, these participants also showed an elevation in the levels of DOPA.

TABLE 8
Difference detection of putative metabolites
using stable isotope dilution multiple reaction
monitoring mass spectrometry (SID-MRM-MS).

| | Fold | Comparison | | |
|---|---|---|---|---|
| Metabolite | Change | Groups | Mode | p-value |
| PC ae C38:4 | ↓ | NC vs Cₚᵣₑ | POS | 0.00417 |
| Proline | ↓ | NC vs Cₚᵣₑ | POS | 3.00E−05 |
| Lysine | ↓ | NC vs Cₚᵣₑ | POS | 0.0020 |
| Serotonin | ↓ | NC vs Cₚᵣₑ | POS | 0.0160 |
| Taurine | ↓ | NC vs Cₚᵣₑ | POS | 0.0030 |
| DOPA | ↑ | NC vs Cₚᵣₑ | POS | 0.0001 |
| Phenylalanine | ↓ | NC vs Cₚᵣₑ | POS | 1.00E−05 |
| Acylcarnitine | ↓ | NC vs aMCI/AD | POS | 0.0001 |
| C7-DC | | | | |

Up arrows in Table 8 indicate up-regulation in the comparison group as compared to the normal control (NC) participants while the down arrow suggests down-regulation in these groups.

The targeted meta bolomic/lipidomic analysis identified of a set of ten metabolites, comprised of PCs, lyso PCs, and ACs that were depleted in the plasma of the Cₚᵣₑ participants compared to the NC group. These metabolites remain depleted after the same participants phenoconverted to aMCI/AD (Cₚₒₛₜ) and were nearly equivalent to the low levels seen in the cognitively impaired aMCI/AD group. A simple logistic model with the ten metabolite panel was used to predict/classify Cₚᵣₑ and NC participants. When displayed as a ROC curve, the ten metabolite panel comparing Cₚᵣₑ and NC participants yielded an AUC of 0.96, while the panel yielded an AUC of 0.827 for the aMCI/AD vs NC classification.

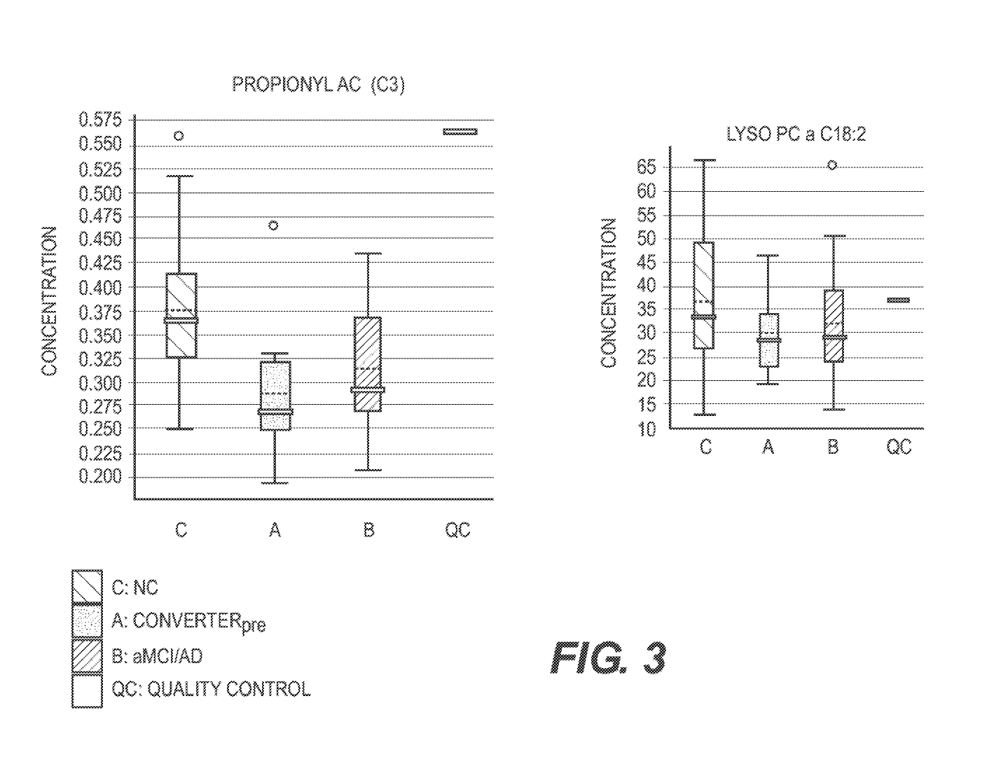

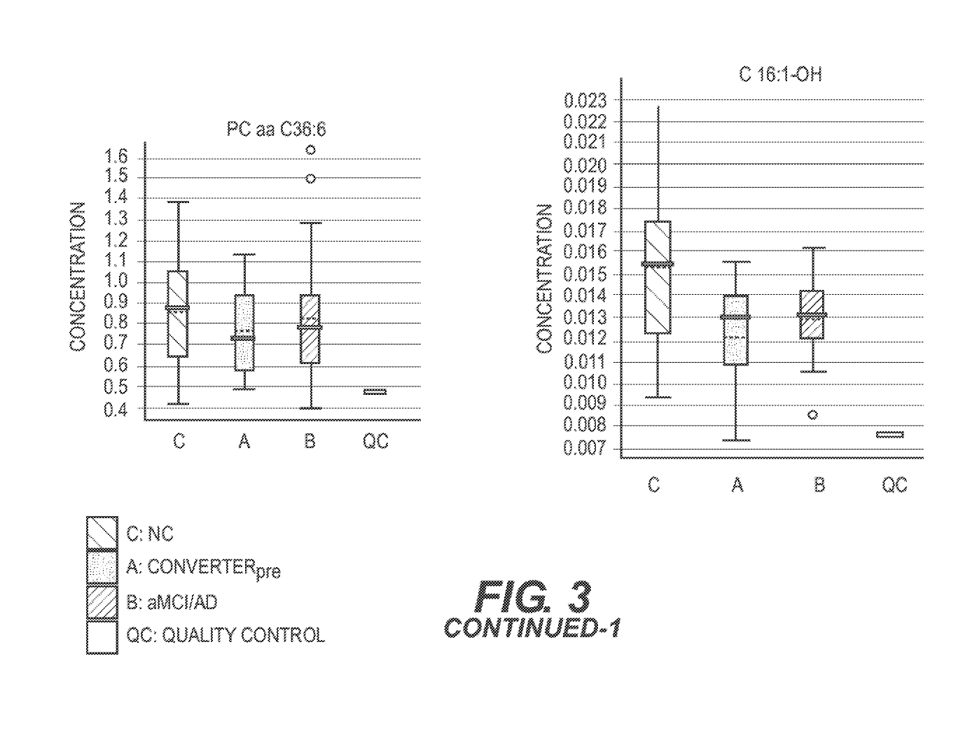

To confirm the reproducibility of the ten metabolite panel from the discovery samples, targeted quantitative metabolomics/lipidomics analyses was performed using plasma from the independent validation group of 40 participants as an independent cross validation. One sample from the MCI/AD group was not available for lipidomic analysis. The validation samples were obtained from the last available visit from the aMCI/AD, Cₚᵣₑ and NC groups and were designated as groups A, B, and C and analyzed in a blinded fashion, without specification of diagnostic identities. The samples were processed and analyzed using the same SID-MRM-MS technique as in the discovery phase. The blinded data were statistically analyzed to determine if the unknown groups could be characterized into the correct diagnostic categories based solely on the levels of the ten metabolite panel. The validation analysis revealed lower levels of the assayed metabolites in groups A and B compared to group C (FIG. 3). Based on these quantitative differences, it was predicted that group C was the NC group and groups A and B were Cₚᵣₑ and aMCI/AD groups, respectively. Subsequent un-blinding of the groups confirmed these predictions.

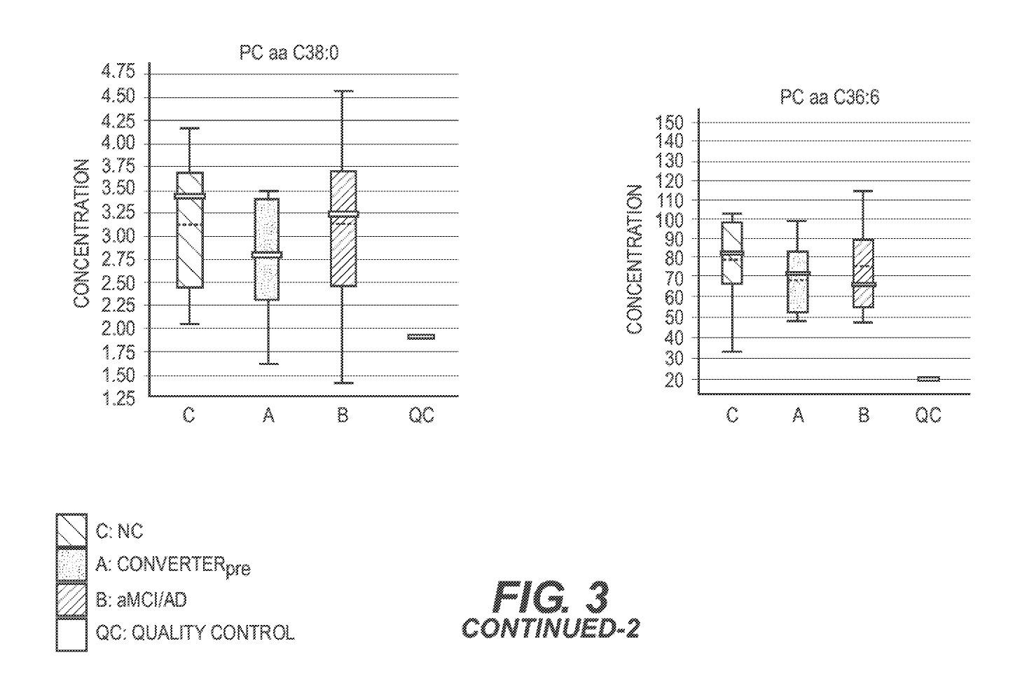

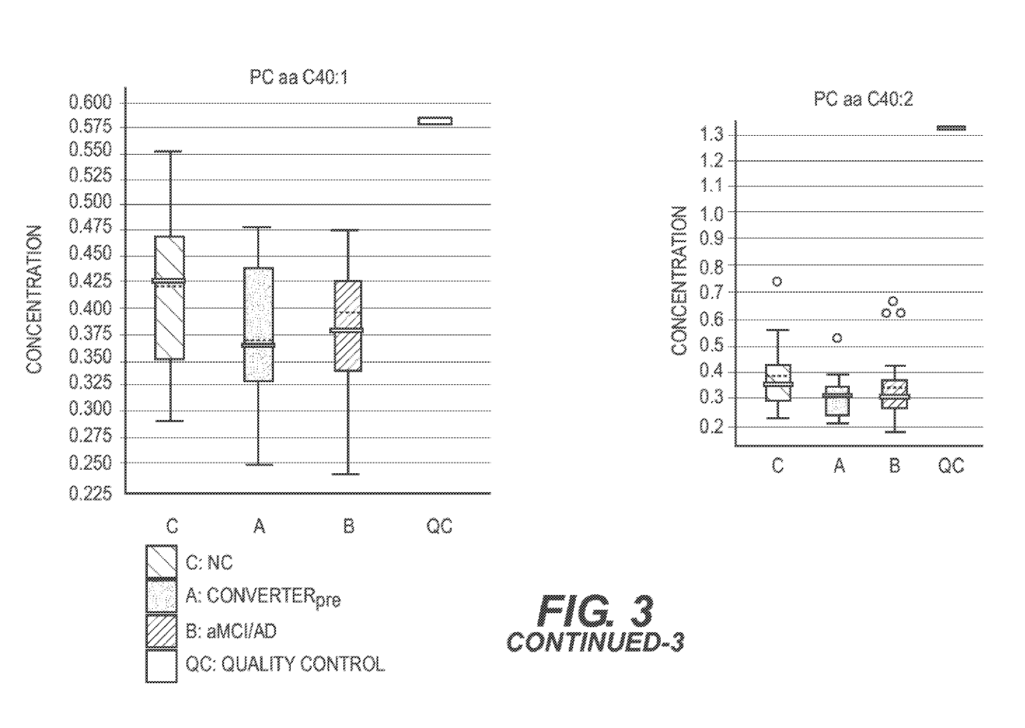

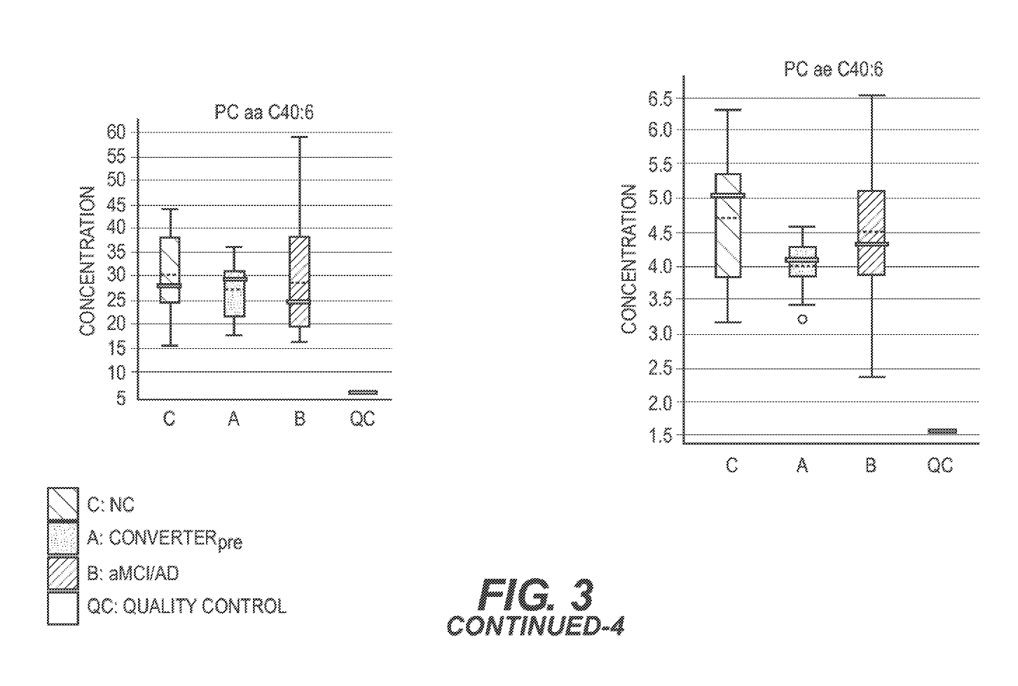

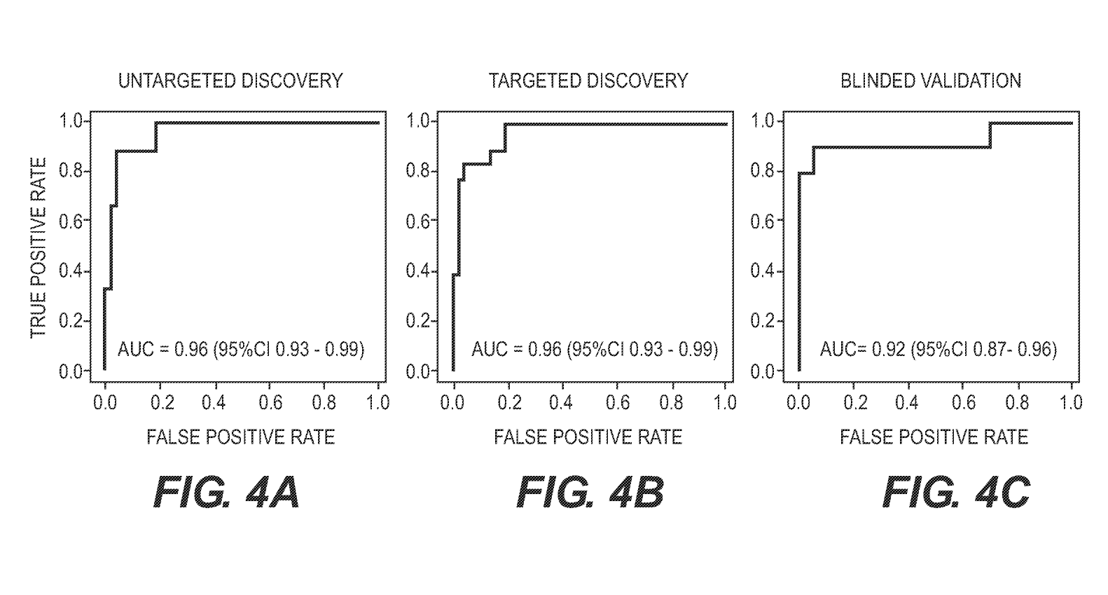

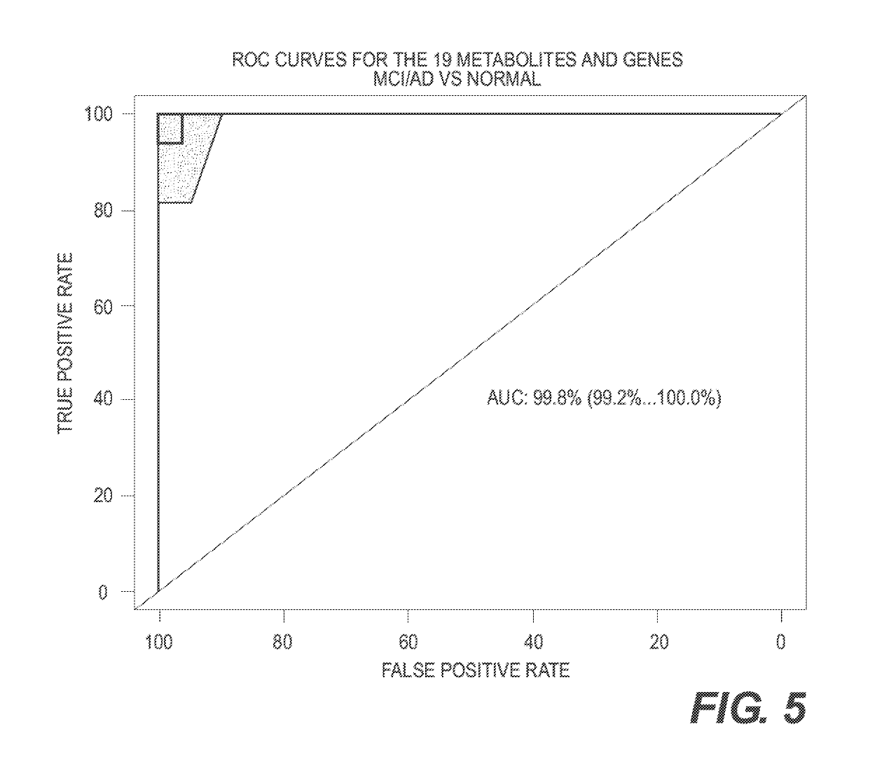

The meta bolomic data was used from the untargeted LASSO analysis to build separate linear classifier models that would distinguish the aMCI/AD group from the NC group and the Cₚᵣₑ group from the NC group. A receiver operating characteristic (ROC) analysis was employed to determine the area under the ROC curve (AUC) to assess the performance of the classifier models in differentiating the groups. When distinguishing between the Cₚᵣₑ and NC groups, the LASSO identified metabolites yielded an AUC of 0.96 (FIG. 4a), and 0.83 when distinguishing the aMCI/AD and NC groups. These high AUC values demonstrate robust discrimination between the aMCI/AD, Cₚᵣₑ, and NC groups by the models. Using the same linear classifier method, a simple logistic model was constructed using just the ten metabolite panel and used this to classify the same groups. When displayed as a ROC curve, the ten metabolite panel classified Cₚᵣₑ and NC participants with an AUC of 0.96 (FIG. 4b), while the panel yielded an AUC of 0.827 for the aMCI/AD vs NC classification.

The effects of apolipoprotein epsilon (ApoE) genotype was considered. ApoE is involved in lipid metabolism and is a known risk factor for Alzheimer's Disease and ApoE genotype status was accounted for by repeating this classification analysis with APO-ε4 presence as a covariate and it was found the classification accuracy changed only minimally from 0.96 to 0.968 (p=0.992). Furthermore, a classifier model using only APO-ε4 produced an AUC of 0.54 for classifying the Cₚᵣₑ and NC groups implying virtually random classification. These findings clearly indicate that the presumed pathophysiology reflected by the ten metabolite biomarker panel is independent of ApoE mediated effects. Finally, the same simple logistic classifier model developed for the discovery samples was applied to the independent validation samples. The ROC constructed from the validation group data classified Cₚᵣₑ and NC participants with an AUC of 0.92 and 0.77 for classifying the aMCI/AD vs NC groups. For a specificity of 90%, the ten metabolite panel yielded a sensitivity of 83.3% for correct classification of the Cₚᵣₑ and NC participants in the discovery phase and a sensitivity of 90% in the validation phase.

### Example 3

### Sample Extraction Methods for Gene Expression Analysis

When blood was drawn from the subject for lipidomic analysis according to Example 2 above, blood was also drawn and placed in a PAXgene® blood tube (Qiagen). Samples were then processed according to the manufacturer's suggested protocol for RNA extraction.

Messenger RNA (mRNA) sequencing was performed using an Illumina High Seq™ sequencing platform. In brief, after specimen thawing, globin mRNA was depleted from the total RNA samples using the GLOBINclear-Human Kit™ (# AM1980, Life Technologies, Grand Island, N.Y., USA), as described by the vendor. A total of 1.25 μg of RNA isolated from whole blood was then combined with biotinylated capture oligonucleotides complementary to globin mRNAs. The mixture was incubated at 50° C. for 15 minutes to allow duplex formation. Streptavidin magnetic beads were added to each specimen, and the resulting mixture was incubated for an additional 30 minutes at 50° C. to allow binding of the biotin moieties by Streptavidin. These complexes, comprising Streptavidin magnetic beads bound to biotinylated capture oligonucleotides that are specifically hybridized to the specimen globin mRNAs, were then separated from the specimen using a magnet. The globin-depleted supernatant was transferred to a new container and further purified using RNA binding beads. The final globin mRNA-depleted RNA samples were quantified using a NanoDrop ND-8000™ spectrophotometer (Thermo Fisher Scientific, Inc., Waltham, Mass., USA).

Libraries were prepared for RNA-Seq using the TruSeq RNA Sample Prep Kit™ (Illumina, Inc., San Diego, Calif., USA), including the use of Illumina in-line control spike-in transcripts. Prior to library preparation, RNA samples were quantitated by spectrophotometry using a NanoDrop ND-8000™ spectrophotometer, and assessed for RNA integrity using an Agilent 2100 BioAnalyzer™ (Agilent Technologies Inc., Santa Clara, Calif., USA) or Caliper LabChip GX™ (PerkinElmer, Waltham, Mass., USA). RNA samples with A260/A280 ratios ranging from 1.6-2.2, with RIN values 7.0, and for which at least 500 ng of total RNA proceeded to library preparation.

Library preparation was initiated with 500 ng of RNA in 50 μl of nuclease-free water, which was subjected to poly(A)+purification using oligo-dT magnetic beads. After washing and elution, the polyadenylated RNA was fragmented to a median size of ˜150 bp and then used as a template for reverse transcription. The resulting single-stranded cDNA was converted to double-stranded cDNA; ends were repaired to create blunt ends, and then a single A residue was added to the 3′ ends to create A-tailed molecules. IIlumina indexed sequencing adapters were then ligated to the A-tailed double-stranded cDNA. A single index was used for each sample. The adapter-ligated cDNA was then subjected to PCR amplification for 15 cycles. This final library product was purified using AMPure™ beads (Beckman Coulter, Inc., Pasedena, Calif., USA), quantified by qPCR (Kapa Biosystems, Inc., Wilmington, Mass., USA), and its size distribution assessed using an Agilent 2100 BioAnalyzer or Caliper LabChip GX.™ Following quantitation, an aliquot of the library was normalized to 2 nM concentration and equal volumes of specific libraries were mixed to create multiplexed pools in preparation for IIlumina sequencing.

RNA-Seq analysis included the data files FASTQ, BAM, translated CEL, quality control and summary. Transcript level differentially expressed gene (DEG) analysis, using the BAM files for input, was conducted using EdgeR™ package in Bioconductor as described in Robinson, M. D., et al. Bioinformatics 26, 139-140 (2010), which is incorporated by reference. A General Linear Model was used in EdgeR™ to compare groups of samples with multiple testing corrections performed using FDR with significance threshold set at 0.1 (10% FDR). Log 2 transformed read counts for differentially expressed transcripts were further analyzed at the gene level. Hierarchical clustering of DEGs, Heatmaps, and PCA analyses were performed using the TM4 software package as described in Saeed, A. I., et al., Methods Enzymol 411, 134-193 (2006), which is incorporated by reference. DEGs were subjected to downstream systems biology analysis using pathway enrichment analysis, Gene Ontology enrichment, and gene network enrichment analysis based on the Fisher's exact test (Ingenuity IPA [Ingenuity® Systems, www.ingenuity.com] as described in Jimenez-Marin, A., et al., BMC Proceedings 3 Suppl 4, S6 (2009) which is incorporated by reference, and Pathway Studion™ software packages [Elsevier, www.elsevier.com] as described Nikitin, A., et al., Bioinformatics 19, 2155-2157 (2003) which is incorporated by reference. In addition variant analysis was performed as described in Wang, K., et al., Nucleic Acids Res 38, e164 (2010), which is incorporated by reference. Sample classification was performed using R based machine learning algorithms of Support Vector Machine with recursive feature elimination (SVM-RFE) and 2-fold cross validation as described in Guyon, I., et al., Machine Learning 46, 389-422 (2002), which is incorporated by reference.

For each group comparison a minimal number of features that provided maximum accuracy of classification (as determined by SVM-RFE) was used to generate Receiver Operating Characteristic (ROC) curves. ROC curves were generated for each data type with 95% confidence intervals using the R package pROC as described in Robin, X., et al, BMC Bioinformatics 12, 77 (2011) which is incorporated by reference: an open-source package for R to analyze ROC curves (Bioconductor). Leave-one-out cross validation was used to validate the results of ROC analysis and the bootstrapping option was used to generate confidence intervals. Overfitting can be a significant problem when global profiling data are used to classify samples. In this analysis this problem was addressed by applying a multi-step data reduction, feature ranking, and various cross-validation procedures to each dataset. First, data was pre-filtered on significance of differences, which led to a significant reduction in the number of features. Second, we the RFE algorithm was applied in conjunction with SVM for each group comparison that allowed ranking the features and selecting a minimal number of features allowing for maximum classification accuracy. SVM-RFE algorithm has been reported in the literature as one of the best classification algorithms for addressing overfitting issues in gene expression analysis. See Guyon, I., et al, Machine Learning 46, 389-422 (2002), which is incorporated by reference. For each data type this algorithm was applied with rigorous cross-validation procedures: at each step in SVM-RFE a 2-fold cross-validation was used with 10,000 permutations (a variation of k-fold cross-validation). For each fold, data points were randomly assigned to two sets, d0 and d1 (which were implemented by shuffling the data array and then splitting it in two), which were then used to train on d0 and test on d1, followed by training on d1 and testing on d0. This 2-fold cross-validation method has the advantage that the training and test sets are both large compared with k-fold cross-validation, and each data point is used for both training and validation on each fold as described by Arlot, S and Cellise, A., Statistics Surveys 4, 40-79 (2010) and Picard, R., and Cook, R., Journal of the American Statistical Society 79, 575-583 (1984), which are incorporated by reference. After this step the number of features for each data set was already reduced 5 to 10 fold. Third, the ROC was calculated for each set of minimal number of features (that provided maximum accuracy of classification) and validated using leave-one-out cross-validation procedure. Finally, the confidence intervals for ROC curves were estimated using the bootstrapping approach. Overall, the problem of overfitting was directly addressed in this analysis by multiple computational procedures of feature reduction, ranking, elimination, and cross-validation that were applied consecutively for each dataset.

Differential Gene Expression Analysis

Once the RNA transcripts were sequenced, levels of each transcript were quantified as described above. These levels were then assessed to determine if a specific set of genes in Cₚᵣₑ subjects was differentially expressed compared to normal subjects.

The combination of the lipidomic profile and the gene expression profile were generated to create a combined classifier model. The same statistical regularized learning technique that was utilized for development of the 10 lipid panel (Mapstone, M., et al. Nat Med., 20(4):415-418; doi: 10.1038/nm.3466 (2014), which is incorporated by reference) was also used to discover the panel of differentially expressed genes (DEGs) selected for use in combination with the 10 lipid panel to create the combined classifier model. In brief, using the 10 lipids as constants, the top 120 DEG set was interrogated for statistically significant members that added significance to the combined classifier. The method used a receiver operating characteristic (ROC) regularized learning technique (Ma, S. & Huang, J., Bioinformatics 21, 4356-4362 (2005) and Liu, Z. & Tan, M., Biometrics, 64: 1155-1161 (2008), which are incorporated by reference). The technique is based on the least absolute shrinkage and selection operator (LASSO) penalty (Tibshirani, R., Journal of the Royal Statistical Society, Series B (Methodological 58, 267-288 (1996) and Hastie, T., et al., The Elements of Statistical Learning; Data Mining, Inference, and Prediction, (Springer-Verlag, New York, 2008), which are incorporated by reference). The LASSO penalty is implemented with the R package ‘glmnet’ (Friedman, J., et al., Journal of Statistical Software, 33: 1-22 (2010), incorporated by reference), which uses cyclical coordinate descent in a path-wise fashion. The classification performance of the selected DEGs and 10 lipid set was assessed using area under the ROC curve (AUC). The least number of DEGs that, combined with the 10 lipid panel, provided the most significant AUC values were selected.

What is claimed is:

1. A method of determining if a subject has an increased risk of suffering from memory impairment, the method comprising
a) analyzing at least one sample from the subject to determine a value of the subject's biomarker profile, and
b) comparing the value of the subject's biomarker profile with the value obtained from subjects determined to define a normal biomarker profile, to determine if the subject's biomarker profile is altered compared to a normal biomarker profile,
wherein a change in the value of the subject's biomarker profile is indicative that the subject has an increased risk of suffering from future memory impairment compared to those defined as having a normal biomarker profile.

2. The method of claim 1, wherein the biomarker profile comprises a lipidomic profile, wherein the lipidomic profile comprises acylcarnitines (ACs) or phosphatidyl cholines (PCs).

3. The method of claim 2, wherein the lipidomic profile comprises at least two metabolites selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.

4. The method of claim 3, wherein the lipidomic profile comprises at least three, four, five, six, seven, eight, nine or 10 metabolites selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.

5. The method of claim 1, wherein the subject's biomarker profile comprises a gene expression profile, wherein the gene expression profile comprises expression levels of at least one gene selected from the group consisting of APOBEC3A, ASXL1, CLK4, FAM217B, LYPLA1, OXR1, SCLY, STAG2, and TVP23C-CDRT4.

6. The method of claim 5, wherein the subject's gene expression profile comprises expression levels of APOBEC3A, ASXL1, CLK4, FAM217B, LYPLA1, OXR1, SCLY, STAG2, and TVP23C-CDRT4.

7. The method of any of claims 1-6, wherein the normal biomarker profile comprises the subject's biomarker profile prior to the onset of memory impairment.

8. The method of any of claim 1-6, wherein the normal biomarker profile comprises a biomarker profile generated from a population of individuals that do not presently or in the future display memory impairment.

9. A method of monitoring the progression of memory impairment in a subject, the method comprising
a) analyzing at least two blood samples from the subject with each sample taken at different time points to determine the values of each of the subject's biomarker profiles, and
b) comparing the values of the subject's biomarker profiles over time to determine if the subject's biomarker profile is changing over time,
wherein a change in the subject's biomarker value over time is indicative that the subject's risk of suffering from memory impairment is increasing over time.

10. The method of claim 9, wherein the biomarker profile comprises a lipidomic profile, wherein the lipidomic profile comprises acylcarnitines (ACs) or phosphatidylcholines (PCs).

11. The method of claim 10, wherein the lipidomic profile comprises at least two metabolites selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.

12. The method of claim 9, wherein the subject's biomarker profile comprises a gene expression profile, wherein the gene expression profile comprises expression levels of at least one gene selected from the group consisting of APOBEC3A, ASXL1, CLK4, FAM217B, LYPLA1, OXR1, SCLY, STAG2, and TVP23C-CDRT4.

13. The method of claim 12, wherein the gene expression profile comprises expression levels of APOBEC3A, ASXL1, CLK4, FAM217B, LYPLA1, OXR1, SCLY, STAG2, and TVP23C-CDRT4.

14. A method of monitoring the progression of a treatment for memory impairment in a subject, the method comprising
a) analyzing at least two samples from a subject undergoing treatment for memory impairment with each sample taken at different time points to determine the values of each of the subject's biomarker profiles, and
b) comparing the values of the subject's biomarker profiles over time to determine if the subject's biomarker profile is changing over time in response to the treatment,
wherein a lack of change or a further deviation from a normal biomarker profile in the subject's biomarker profile is indicative that the treatment for memory impairment is not effective, and wherein an approximation of the subject's biomarker profile over time towards a normal biomarker profile is indicative that the treatment for memory impairment is effective in treating memory impairment in the subject.

15. The method of claim 14, wherein the biomarker profile comprises a lipidomic profile, wherein the lipidomic profile comprises acylcarnitines (ACs) or phosphatidylcholines (PCs).

16. The method of claim 15, wherein the lipidomic profile comprises at least two metabolites selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.

17. The method of claim 14, wherein the subject's biomarker profile comprises a gene expression profile, wherein the gene expression profile comprises expression levels of at least one gene selected from the group consisting of APOBEC3A, ASXL1, CLK4, FAM217B, LYPLA1, OXR1, SCLY, STAG2, and TVP23C-CDRT4.

18. The method of claim 17, wherein the gene expression profile comprises expression levels of APOBEC3A, ASXL1, CLK4, FAM217B, LYPLA1, OXR1, SCLY, STAG2, and TVP23C-CDRT4.

19. A method of determining if a subject has an increased risk of suffering from memory impairment, the method comprising analyzing at least one sample from the subject to determine levels of individual biomarkers and comparing the levels of individual biomarkers with the value of levels of the biomarkers in one or more normal individuals to determine if the levels of each biomarker are altered compared to normal levels, wherein a change in the value of the subject's biomarkers is indicative that the subject has an increased risk of suffering from memory impairment compared to a normal individual.

20. The method of claim 19 wherein the biomarkers are genes expression levels of genes selected from the group consisting of APOBEC3A, ASXL1, CLK4, FAM217B, LYPLA1, OXR1, SCLY, STAG2, and TVP23C-CDRT4 and levels of plasma lipids selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.
